SEQUENCE LISTING (8 sequences)

SEQ ID NO: 1 - DNA, 20 bp, Artificial Sequence
  primer (actin-F)
gtgggccgccctaggcacca

SEQ ID NO: 2 - DNA, 20 bp, Artificial Sequence
  primer (actin-R)
tggccttagggttcaggggg

SEQ ID NO: 3 - DNA, 20 bp, Artificial Sequence
  primer (erythropoietin-F (Epo-F))
aatggaggtggaagaacagg

SEQ ID NO: 4 - DNA, 19 bp, Artificial Sequence
  primer (erythropoietin-R (Epo-R))
acccgaagcagtgaagtga

SEQ ID NO: 5 - DNA, 20 bp, Artificial Sequence
  primer (glyceraldehyde 3-phosphate dehydrogenase-F (GAPDH-F))
tggcatcgtggaagggctca

SEQ ID NO: 6 - DNA, 20 bp, Artificial Sequence
  primer (glyceraldehyde 3-phosphate dehydrogenase-R (GAPDH-R))
ggaagaatgggagttgctgt

SEQ ID NO: 7 - DNA, 21 bp, Artificial Sequence
  primer (hepatocyte growth factor-F (HGF-F))
cttggcatccacgatgttcat

SEQ ID NO: 8 - DNA, 21 bp, Artificial Sequence
  primer (hepatocyte growth factor-R (HGF-R))
tggtgctgactgcatttctca

(19) United States
(12) Patent Application Publication
(10) Pub. No.: US 2016/0008386 A1
(43) Pub. Date: Jan. 14, 2016

(54) COMPOSITION AND METHOD FOR INDUCING EPO-MEDIATED HAEMOGLOBIN EXPRESSION AND MITOCHONDRIAL BIOGENESIS IN NONHAEMATOPOIETIC CELL

(71) Applicant: NATIONAL YANG-MING UNIVERSITY, Taipei (TW)

(72) Inventor: Rong-Tsun WU, Taipei (TW)

(73) Assignee: NATIONAL YANG-MING UNIVERSITY, Taipei (TW)

(21) Appl. No.: 14/792,064

(22) Filed: Jul. 6, 2015

Related U.S. Application Data
(63) Continuation of application No. 13/852,669, filed on Mar. 28, 2013.
Application No. 13/852,669 is a continuation-in-part of application No. 12/343,922, filed on Dec. 24, 2008.

(51) Int. Cl. A61K 31/7034

(52) U.S. Cl. CPC A61K 31/7034

(57) ABSTRACT

A composition for inducing erythropoietin (EPO)-mediated haemoglobin (Hb) expression in a nonhaematopoietic cell of a subject is provided. The composition includes a compound represented by formula (I), wherein R is a glycosyl group; and a pharmaceutical acceptable carrier.

### CROSS-REFERENCE TO RELATED APPLICATIONS

This application is a continuation of co-pending U.S. patent application Ser. No. 13/852,669, filed Mar. 28, 2013, which is a continuation-in-part of U.S. patent application Ser. No. 12/343,922, filed on Dec. 24, 2008. The contents of the above cited application is incorporated into the present disclosure by reference herein and made a part of this specification.

### BACKGROUND OF INVENTION

1. Field of Invention

The present invention relates to a composition and a method for inducing haemoglobin expression, mitochondrial biogenesis and autophagy in a subject.

2. Description of Related Art Ischemia causes oxygen deprivation, cell injury and related organ dysfunctions, such as heart failure, stroke, chronic obstructive pulmonary disease, ischemic retinopathy, liver injury, and acute renal failure. Because mitochondrial dysfunction is a key factor in organ ischemic injury, upon loss of oxygen, mitochondrial oxidative phosphorylation rapidly stops, with resulting loss of the major source of ATP production for energy metabolism.

Erythropoietin (EPO) is essential for the regulation of the mass of erythrocytes in response to changes in tissue oxygenation during hypoxia and anaemia. The protective effects of EPO have been demonstrated in various tissues and experimental models of ischemia-induced injury and have been attributed to its effect on nonhaematopoietic metabolic adaptation, inhibition of apoptosis or stimulation of angiogenesis. Recently, EPO has been reported to stimulate cardiac mitochondrial proliferation through the activation of mitochondrial biogenesis, which is mediated by peroxisome proliferator-activated receptor coactivator 1-α (PGC-1α), a key regulator of cardiac bioenergetics. Clinically, EPO reverses cardiac remodeling, improves cardiac function, and enhances the exercise tolerance and quality of life of patients by inducing protective effects beyond the correction of anaemia. These findings highlight the possibility that EPO-mediated protection may depend on its modulatory effects on intracellular energetics.

Haemoglobin (Hb) is the main oxygen transporter in erythrocytes. Its main form, haemoglobin A, is a tetramer consisting of two α- and β-polypeptide chains, each carrying a heme group. Recently, Hb was unexpectedly found to be expressed in many nonhaematopoietic cells, which may facilitate tissue oxygen transport or increase cellular oxygenation to provide an intrinsic protective mechanism against hypoxic/ischemic injury.

Sleep has been implicated in the plastic cerebral changes that underlie learning and memory. Both rapid eye movement (REM) and non-REM sleep (NREM) play important roles in memory. Behavioral observations in rats show that periods of learning are associated with subsequent increases in REM sleep, whereas REM sleep deprivation impairs memory of cognitive procedural or implicit types of material previously learned. NREM was found to be positively correlated with the ability to retain a word pair-association list which was a declarative memory. In addition, the transition from short-term to long-term memories by reactivation of sharp wave-ripples in the hippocampus during NREM was important for memory consolidation. It has also been demonstrated that inducing slow oscillation-like potential fields by transcranial application of oscillating potentials (0.75 Hz) during early nocturnal NREM, enhances the retention of hippocampus-dependent declarative memories in healthy humans.

Patients with dementias, such as Alzheimer's disease (AD), often have nocturnally disrupted sleep. While the REM sleep in early-stage AD patients is relatively unaffected by the disease process, later stages of AD are marked by significant losses of REM sleep. These disruptions of nighttime sleep increase in magnitude with increasing severity of dementia. Memory loss is accompanied by the accumulation of oxidative damage to lipids, proteins, nucleic acids, and by mitochondrial decay, all of which can disrupt neuronal function in aging and disease. Sleep deprivation (SD) also induced oxidative stress which resulted in memory loss and impaired mitochondrial activity. A study showed that 36h-SD in young adults results in neuropsychological results similar to those found in nom al people aged approximately 60 years. Therefore, the regulation of mitochondrial function and ROS homeostasis may be useful as a therapeutic intervention in the oxidative stress-related memory loss.

Moreover, both EPO and the EPO receptor are expressed in neurons and astrocytes, and EPO is produced primarily by astrocytes in the brain. EPO is widely used to enhance erythropoiesis in patients with anemia and recently has been found to have many non-haematopoietic beneficial effects, including cardioprotection and neuroprotection. An early clinical study has demonstrated cognitive improvement during EPO treatment among patients with chronic renal failure. Recently studies have shown that a high-dose EPO treatment improves hippocampal plasticity and cognitive performance in patients suffering from neuropsychiatric diseases. High-dose EPO also enhances hippocampal long term potentiation by modulating plasticity, synaptic connectivity and activity of memory-related neuronal networks and improves operant conditioning stability of cognitive performance in healthy mice.

It is hypothesized that EPO may play a pivotal role for pharmacological applications in the treatment of SD-induced impairment of hippocampal learning and memory by modulating downstream mitochondrial regulator expression. Due to the fact that EPO has limited clinical use because it cannot freely cross the blood-brain barrier (BBB), only systemic dosing of high-dose recombinant Epo (rEpo) would result in neuroprotective activity.

Autophagy or “self digestion process” is an important physiological process that targets cytosolic components such as proteins, protein aggregates and organelles for degradation in lysosomes. The autophagic process is also essential for maintaining neuronal homeostasis, and its dysfunction has been directly linked to an increasing number of diseases. In addition, autophagy is directed to recycling intracellular nutrients in order to sustain cell metabolism during starvation, and eliminating damaged organelles and proteins that have accumulated during stress.

Defective autophagy is a major contributor to diseases which may be, but not limited to, neurodegeneration, liver disease, and cancer. A lot of human neurodegenerative diseases are associated with aberrant mutant and/or polyubiquitinated protein accumulation and excessive neuronal cell death.

Polygonum multiflorum Thunb is a Chinese medicine used for the treatment of anaemia, liver diseases, and other diseases commonly associated with aging. The present invention provides small molecular compounds isolated and identified from Polygonum multiflorum Thunb. These compounds have effects in experimental models of cardiovascular diseases, cerebral ischemia, Alzheimer's disease and inflammation diseases, and have antioxidant and free radical-scavenging properties. In addition, the present invention provides therapeutic effects and physiological mechanisms of such compounds in animal models.

### SUMMARY OF INVENTION

The present invention provides a composition for inducing erythropoietin (EPO)-mediated haemoglobin (Hb) expression in a nonhaematopoietic cell of a subject. The composition comprises a compound represented by formula (I):

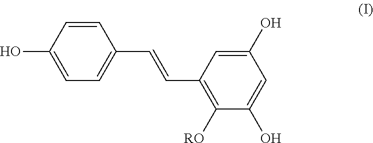

wherein R is a glycosyl group; and a pharmaceutical acceptable carrier.

The glycosyl group is one selected from the group consisting of dihydroxyacetone, glucose, galactose, glyceraldehyde, threose, xylose, mannose, ribose, ribulose, tagatose, psicose, fructose, sorbose, rhamnose, erythrose, erthrulose, arabinose, lyxose, allose, altrose, gulose, idose, talose, sucrose, lactose, maltose, lactulose, trehalose, cellobose, isomaltotriose, nigerotriose, maltotriose, melezitose, maltotriulose, raffinose, kestose and a combination thereof.

In accordance with the present invention, the compound induces Hb-α, Hb-β, or dimeric Hb expression in the nonhaematopoietic cell of the subject, enhances erythropoietin-erythropoietin receptor binding affinity and also binds to the erythropoietin-bound erythropoietin receptor complex. In addition, the compound enhances endogenous EPO expression and stimulates Hb expression in the nonhaematopoietic cell.

The nonhaematopoietic cell is selected from the group consisting of a renal cell, a hepatocyte, a cardiomyocyte, a myoblast, a glial cell, a neuronal cell and a retinal pigment epithelium cell.

The present invention further provides a method for inducing erythropoietin (EPO)-mediated haemoglobin (Hb) expression in a nonhaematopoietic cell of a subject, comprising administering to the subject a therapeutically effective amount of the aforementioned compound of formula (I). In accordance with the present invention, the subject suffers a disease or syndrome selected from the group consisting of hypoxia, anaemia, renal ischemia, myocardial ischemia, lung ischemia, neurodegenerative disease, neuropsychiatric disease, age-related macular degeneration (AMD)-related disease and a combination thereof.

The present invention further provides a composition for inducing erythropoietin (EPO)-mediated mitochondrial biogenesis in a nonhaematopoietic cell of a subject, comprising the aforementioned compound of formula (I) and a pharmaceutical acceptable carrier.

In accordance with the present invention, the compound induces an increase of a mitochondrial number or PGC-1α expression for inducing the EPO-mediated mitochondrial biogenesis, enhances erythropoietin-erythropoietin receptor binding affinity and also binds to the erythropoietin-bound erythropoietin receptor complex. In addition, the compound enhances endogenous EPO expression and stimulates Hb expression in the nonhaematopoietic cell of the subject. The EPO-mediated mitochondrial biogenesis is PGC-1α-dependent.

The nonhaematopoietic cell is selected from the group consisting of a renal cell, a hepatocyte, a cardiomyocyte, a myoblast, a glial cell, a neuronal cell and a retinal pigment epithelium cell.

The present invention further provides a method for inducing erythropoietin (EPO)-mediated mitochondrial biogenesis in a nonhaematopoietic cell of a subject, comprising administering to the subject a therapeutically effective amount of the aforementioned compound of formula (I). The compound induces an increase of a mitochondrial number or PGC-1α expression for inducing the EPO-mediated mitochondrial biogenesis.

The subject suffers a disease or syndrome selected from the group consisting of hypoxia, anaemia, ischemia-related disease, neurodegenerative disease, neuropsychiatric disease, age-related macular degeneration (AMID)-related disease, cardiomyopathy, brain aging, chronic liver disease, multiple sclerosis, Pompe disease, hypertension, cardiac failure, obesity, diabetes mellitus, renal disease, atherosclerosis, aging, metabolic syndrome and a combination thereof.

The ischemia-related disease is one selected from the group consisting of heart ischemia, ischemic neurodegeneration, brain ischemia, myocardial ischemia, limb ischemia, cerebral ischemia, hepatic ischemia, retinal ischemia, stroke, nephritic ischemia, pulmonary ischemia, intestinal ischemia, cardiovascular ischemia, renal ischemia and kidney ischemia. The neurodegenerative disease is one selected from the group consisting of Alzheimer's disease, Parkinson's disease and Huntington's disease.

The present invention further provides a method for inducing autophagy in a subject having an autophagy defect, comprising administering to the subject a therapeutically effective amount of the aforementioned compound of formula (I), wherein the autophagy enhances clearance of protein aggregates in the subject.

The autophagy defect is in a cell expressing the protein aggregates in the subject, wherein the protein aggregate is an aggregate selected from the group consisting of hungtingtin, amyloid β (Aβ), α-synuclein, tau, superoxide dismutase 1 (SOD1), variants and mutated forms thereof, and a combination thereof. The cell of the subject is a neuronal cell or a glial cell.

The autophagy defect is one disease selected from the group consisting of neurodegenerative disease, retinal disease, Crohn's disease, aging, cardiac hypertrophy, chronic heart failure, tuberculosis, chronic obstructive pulmonary disease (COPD), cystic fibrosis, hepatic steatosis, polycystic kidney disease, renal failure, muscle atrophy, Paget's disease of bone, inclusion body myopathy, fronto-temporal dementia, glomerular disease, metabolic disease, glycogen storage disease type II, inflammatory bowel disease, and Pompe disease. The neurodegenerative disease is one selected from the group consisting of Huntington's disease, Alzheimer's disease, Parkinson's disease, amyotrophic lateral sclerosis (ALS) and insomnia.

The present invention further provides a composition for inducing autophagy in a subject having an autophagy defect. The composition comprises the aforementioned compound of formula (I) and a pharmaceutical acceptable carrier.

In addition, the invention provides a method for preventing memory loss in a subject, comprising administering to the subject a therapeutically effective amount of the aforementioned compound of formula (I). The compound induces erythropoietin (EPO) to activate the autophagy in the subject.

The autophagy enhances protein clearance in the subject.

The autophagy defect is a neurodegenerative disease selected from the group consisting of Huntington's disease, Alzheimer's disease, Parkinson's disease and insomnia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A to FIG. 1B show EH-201 characterization. (A) HPLC profile of EH-201. Mightysil RP-18 column (4.6×250 mm i.d., 5 μm) was used at flow rate of 0.8 ml/min with MeOH/H₂O (20/80, v/v) gradient to 100% MeOH in 60 minutes in the detection wavelength of 280 and 300 nm. (B) Positive ion mode LC-APCUMS/MS of EH-201.

FIG. 2A to FIG. 2J show that EH-201 is a potent inducer of EPO expression. (A) The chemical structure of EH-201. (B, C) The EH-201-treated kidney slices and hepatocytes were analyzed for EPO expression by Q-PCR and Western blotting. (D, E) Primary mice cardiomyocytes and (F, G) C2C12 myotubes were treated with EH-201, and the effects on EPO and EPOR expression were analyzed by QPCR and Western blotting. (H) The bone marrow cells were incubated with EH-201 for 48 h, and the expression of EPO was detected by Q-PCR. (I) The bone marrow cells were incubated with EH-201, and the colonies were counted on day 9 for burst-fon ling units-erythroid (BFU-E). (J) The quantification of the differentiated erythroid progenitors was performed using a haemoglobin colorimetric assay. The control represents vehicle treatment. The values are presented as the means±SEM (n=6 for each). *P<0.01, *P<0.05 versus control, Student's t-test.

FIG. 3A to FIG. 3G show that the induction of mitochondrial biogenesis by EH-201 is mediated by EPO. (A, B) EH-201-treated kidney slices and primary cardiomyocytes and (C, D) EH-201-treated hepatocytes and C2C12 myotubes with or without the neutralizing EPO antibody (nEPO-ab, 1 μg/ml) were analyzed for PGC-1α, expression by QPCR (n=6) and Western blotting (n=4), citrate synthase activity (n=3), and mtDNA copy number (n=6) and via the MitoTracker assay (n=6). The control represents vehicle treatment. (E, F and G) rhEPO was given to kidney slices, hepatocytes and C2C12 myotubes. The mitochondrial activity was determined by PGC-1α Q-PCR (n=6), citrate synthase activity (n=3), mtDNA copy number (n=6), and MitoTracker assays (n=6). The control represents vehicle treatment. PGC-1α siRNA-transfected C2C12 myotubes were treated with rhEPO (n=6). The control represents the scrambled siRNA treatment. The values are presented as the means±SEM. **P<0.01, *P<0.05 versus untreated control, n.s., not significant, Student's t-test.

FIG. 4A to FIG. 4I show that the induction of haemoglobin expression in nonhaematopoietic cells by EH-201 is mediated by EPO. (A) Cultured C2C12 myotubes under normoxia or hypoxia (5% O₂) for 24 hours were analyzed for the expression of haemoglobin-alpha (Hb-α) and -beta (Hβ) by RT-PCR, followed by 1.5% agarose gel electrophoresis. (B) The rhEPO-treated C2C12 myotubes with or without PGC-1α siRNA transfection were analyzed for haemoglobin expression by Q-PCR (n=6). The control represents the scrambled siRNA treatment. (C, D) The rhEPO-treated kidney slices and hepatocytes were analyzed for haemoglobin expression by Q-PCR (n=6). The control represents vehicle treatment. (E, F) EH-201-treated kidney slices and primary mice cardiomyocytes and (G, H) EH-201-treated hepatocytes and C2C12 myotubes with or without the neutralizing EPO antibody (nEPO-ab, 1 μg/ml) were analyzed for haemoglobin expression by Q-PCR (n=6). The values are represented as means±SEM. **P<0.01, *P<0.05 versus untreated control, #P<0.05 versus rhEPO treated control (50 ng/ml). (I) The effects of rhEPO and EH-201 on the proliferation of TF-1 cells were determined by a trypan blue dye exclusion assay (upper part of FIG. 4I, n=6). The rhEPO (2 ng/ml) and EH-201 cotreated TF-1 cells were incubated with or without the neutralizing antibody (nEPOR-ab, 0.5 μg/ml; nEPO-ab, 1 μg/ml) for a 48 hour proliferation assay (lower part of FIG. 4I, n=6). The values are represented as means±SEM. **P<0.01, *P<0.05 versus control (upper part of FIG. 4I) or rhEPO alone (lower part of FIG. 4I), ##P<0.01 versus rhEPO+ EH-201 25 M, Student's t-test.

FIG. 5A to FIG. 5G show that EH-201 increases endurance performance and activation of mitochondrial activity and haemoglobin expression in mice. (A) The endurance of normal mice was measured with the rotarod exercise under normoxic or hypoxic (8% O₂) conditions (ND: normal diet). (B) The effect of EH-201 on plasma RBC numbers and haemoglobin levels. (C, D) EPO mRNA expression in the kidney and liver of mice was measured by Q-PCR after 3 days of EH-201 administration. The serum levels of EPO were determined by ELISA. (E, F) Isolated myocardium tissues after 3 days of EH-201 administration were analyzed for haemoglobin expression by Q-PCR, and the mitochondrial biogenesis was determined by mtDNA copy number. (G) The effects of EH-201 treatment on ventricular haemoglobin (Hb) expression were quantified by TMBZ staining in SDS-PAGE (left part of FIG. 5G). The quantification of Hb expression (tetramer and dimer, right part of FIG. 5G). The values are represented as the means±SEM (n=5 animals each group). **P<0.01, *P<0.05 versus the ND group; ##P<0.01, #P<0.05 versus the day 7 ND group by one-way ANOVA with Tukey's posthoc test.

FIG. 6A to FIG. 6H show that EH-201 has therapeutic effects on cardiac dysfunction in doxorubicin (Dox)-induced cardiomyopathy in mice. (A) The survival rate was analyzed using the Kaplan-Meier method (detailed treatment protocol in Materials and Methods). The normal (N) group represents saline injection. (B) The effect of EH-201 treatment on mice performing the hypoxic rotarod endurance test two weeks after Dox injection. (C, D) The effect of EH-201 on cardiac abnormality and functionality was characterized by ECG and echocardiography. EF, ejection fraction; FS, fractional shortening; LVIDs/d, left ventricular internal diameter at systole/diastole. (E) Representative photomicrographs of left ventricular sections of mouse hearts stained with haematoxylin-eosin and Masson's trichrome (left part of FIG. 6E, bars=10 μm). The blue staining indicates fibrosis, and quantification of the interstitial fibrosis was performed (right part of FIG. 6E). (F) Isolated myocardium tissues after 2 weeks of Dox were analyzed for haemoglobin expression by Q-PCR and (G) a TMBZ stain of each myocardium lysate of the treatment groups in SDS-PAGE was performed, with (H) quantitative values. The values are represented as the means±SEM (n=5-6 animals each group). **P<0.01, *P<0.05 versus Dox group by one-way ANOVA with Tukey's posthoc test.

FIG. 7A to FIG. 7H show that EH-201 accelerates the recovery from anaemia and renal function in cisplatin-induced nephropathy in mice. (A) Schematic diagram protocol. (B) The time course kinetics of the RBC numbers in the peripheral blood. (C) The time course kinetics of the blood urea nitrogen (BUN) values after cisplatin injection. (D) The functional recovery of the kidneys of mice treated with EH-201 on day 28. (E) The haematoxylin-eosin stain of kidney sections after EH-201 administration on day 28 (bars=100 μm). (F, G) The EPO expression in the kidney and liver on day 28 was determined by Q-PCR. (H) The numbers of BFU-E colonies in the isolated bone marrow cells from the treated mice on day 28. The values are represented as the means±SEM (n=5-6 animals each group). ##P<0.01 versus with normal group; **P<0.01, *P<0.05 versus control group by one-way ANOVA with Tukey's posthoc test.

FIG. 8 shows that EH-201 induces Sirt1 expression. Sirt1 protein expression in the lysates of the EH-201-treated kidney slices and hepatocytes were analyzed by Western blotting (n=4). The control represents vehicle treatment. The values are represented as the means±SEM. **P<0.01, *P<0.05 compared with control.

FIG. 9 shows ribbon diagrams of the computational docking results for EH-201 on EPO/EPOR complex. Docking calculations were carried out using DockingServer on EPO complexed with extracellular domain of EPOR protein model (PDB entry code 1cn4). The carbon backbone (green color) with balls and sticks indicated the ligand molecule EH-201, the helix (red color, left part of FIG. 9) indicated the helix A of EPO, and the loop (gray color, right part of FIG. 9) indicated the loop 5 of EPOR. The predictive interaction residues including PRO¹⁴⁴, GLU¹⁴⁷, PRO¹⁴⁹, Met¹⁵⁰, and THR¹⁵¹ are located in loop 5 of EPOR, which is important for EPO binding.

FIG. 10A to FIG. 10C show that EH-201-induced EPO production does not involve Hif-a activation. (A) The hypoxia response element (HRE)-driven luciferase reporter (Luci) transfected HEK 293 cells were incubated with EH-201 under normoxia or hypoxia (5% O₂, as the positive control) for 24 hours. The plasmid for β-Galactosidase (β-Gal) was used as a transfection control, and the pGL3-v served as a vector control. Similar results were observed in three additional independent experiments. (B) The VEGF expression of the EH-201-treated hepatocytes were analyzed by Q-PCR (n=3). Hypoxia condition served as a positive control. (C) The Hif-2α protein expression levels in the nuclear lysates of the EH-201-treated kidney slices were analyzed by Western blotting (H: 5% O₂ hypoxia as a positive control). The control represents vehicle treatment. The values are represented as the means±SEM. **P<0.01, *P<0.05 compared with normoxia, Student's t-test.

FIG. 11A and FIG. 11B show that EH-201 increases mitochondrial function and biogenesis in the liver and skeletal muscle. (A, B) Isolated liver and skeletal muscle tissues after 14 days of EH-201 administration were analyzed for the mitochondrial activity by PGC-1α Q-PCR, citrate synthase activity and mtDNA copy number. The values are represented as the means±SEM (n=5 animals each group). **P<0.01, *P<0.05 versus the ND group; ##P<0.01, #P<0.05 versus the day 7 ND group by one-way ANOVA with Tukey's posthoc test.

FIG. 12A and FIG. 12B show that EH-201 has therapeutic effects on cardiac dysfunction in doxorubicin (Dox)-induced cardiomyopathy in mice. (A) The effect of EH-201 on the body weight of mice two weeks after Dox injection. (B) The effect of EH-201 on cardiac function was characterized by ECG, heart rate presented as the beat per second (bps). The values are represented as the means±SEM (n=5-6 animals each group). **P<0.01, *P<0.05 versus Dox group by one-way ANOVA with Tukey's posthoc test.

FIG. 13A to FIG. 13F show that EH-201 stimulats EPO expression in primary astrocytes and PC12 neuronal cells. (A) Structure of EH-201. (B, C) Real time PCR shows that EH-201 treatment for 24 hours increase EPO mRNA in astrocytes and PC 12 neuronal cells. The expression of GAPDH was used as an internal control. (D) Western blotting shows that EH-201 treatment for 24 hours increase EPO protein expression in astrocytes and PC12 neuronal cells. The results are expressed as the relative index of untreated controls ±SD of at least three independent measurements. *P<0.05, **P<0.01 compared to untreated controls by one-way ANOVA followed by Tukey's multiple comparison test. (E) Real time PCR shows that EPO treatment for 24 hours does not increase Hb-α mRNA in astrocytes and PC12 neuronal cells. (F) Real time PCR shows that EH-201 treatment for 24 hours does not increase Hb-a mRNA in astrocytes and PC12 neuronal cells. The expression of GAPDH was used as an internal control. The results are expressed as the relative index of untreated controls±SD of at least three independent measurements. *P<0.05, **P<0.01 compared to untreated controls by one-way ANOVA followed by Tukey's multiple comparison test.

FIG. 14A to FIG. 14F show that EH-201, a neuronal EPO inducer, stimulates the expression of the mitochondrial regulator (PGC-1α, Hb-β) and an antioxidant gene (HO-1) in primary astrocytes and PC12 neuronal cells. (A) Real time PCR shows that EPO or EH-201 treatment for 24 h increase PGC-1α, (B) Hb-β and (C) HO-1 mRNA expression in primary astrocytes. (D) Real time PCR shows that EPO or EH-201 treatment for 24 h increase PGC-1α, (E) Hb-β and (F) HO-1 mRNA expression in PC12 neuronal cells. The expression of GAPDH was used as an internal control. The results are expressed as the relative index of untreated controls±SD from at least three independent measurements. *P<0.05, **P<0.01 by one-way ANOVA followed by Tukey's multiple comparison test.

FIG. 15A to FIG. 15H show that EH-201 increases mitochondrial activity, decreases intracellular ROS and attenuates H₂O₂-induced cell toxicity in primary astrocytes and PC 12 neuronal cells. (A, E) Different forms of Hb (monomer: 16 kD, dimer 32 kD, tetramer: 64 kD) expression identify by Hb-β Ab in primary astrocytes and PC12 neuronal cells treated with EH-201. The results are expressed as the relative expression of untreated controls ±SD from at least three independent measurements. *P<0.05, **P<0.01 by Student's t-test. (B, F) Succinate dehydrogenase activity of astrocytes and PC12 cells treated with EPO or EH-201 at 24 hour is determined using the MTT reduction assay (n=8) and is expressed relative to the respective control conditions (without treatment at 24 hour). The values are the means±SD (n=8). *P<0.05, **P<0.01 compared to untreated controls. (C, G) Astrocytes and PC12 cells treated with EPO or EH-201 for 24 hours are exposed to 100 μM H₂O₂ for 6 hours. Intracellular ROS formation is measured using the DCFH-DA assay. The graph shows results in relative fluorescence units (RFU). The values are the means±SD (n=8). ##P<0.01 compared to untreated controls; *P<0.05, **P<0.01 compared to H₂O₂ controls. Scale bar: 50 μm. (D, H) Astrocytes and PC12 cells treated with EPO or EH-201 for 24 hours are exposed to 500 μM H₂O₂ for 6 hours. Cytotoxicity is analyzed with trypan blue. The values are the means±SD (n=3). ##P<0.01 compared to untreated controls; *P<0.05, **P<0.01 compared to H₂O₂ controls using Student's t-test.

FIG. 16A to FIG. 16F show that EPO is required for the neuroprotective effects of EH-201 in astrocytes and PC12 neuronal cells. (A, D) Co-incubation of EH-201 with an anti-EPO antibody results in the loss of EH-201-induced increase in succinate dehydrogenase activity, as assessed by the MTT reduction assay (n=8) in astrocytes and PC12 cells. *P<0.05, **P<0.01 compared to controls. (B, E) Co-incubation of EH-201 with an anti-EPO antibody for 24 hours results in the loss of EH-201-mediated reduced ROS generation induced by H₂O₂, as assessed by the DCFH-DA assay (n=8), and (C, F) the reduced H₂O₂-mediated cytotoxicity as assessed by trypan blue staining (n=3), *P<0.05, **P<0.01 compared to H₂O₂, ##P<0.01 compared to control using Student's t-test.

FIG. 17A to FIG. 17G show that effects of EH-201 in a mouse model of sleep deprivation-induced memory loss. (A) Procedure of EH-201 treatment in sleep-deprived (SD) mice. (B) Real time PCR and (C) western blot analysis of EPO expression in mouse hippocampus from each group. **p<0.01 statistically significant compared with the SD group; ##p<0.01, statistically significant compared with the control groups. (D) Real time PCR analysis of Hbβ, PGC-1α and HO-1 expression levels in mouse hippocampus (n=6) *p<0.05, **p<0.01 statistically significant compared with the SD group and #p<0.05, ##p<0.01 statistically significant compared with the control by one-way ANOVA followed by Tukey's multiple comparison test. (E) The MTT assay is used as a marker for mitochondrial activity. The values depict mitochondrial function after sleep deprivation of untreated control mice and EH-201-treated mice. *p<0.05, **p<0.01 statistically significant compared with the SD group; ##p<0.01, statistically significant compared with the control groups by one-way ANOVA followed by Tukey's multiple comparison test. (F) Acquisition of step-through passive avoidance during 5 successive training trials in mice treated with or without EH-201. EH-201 treatment does not affect learning ability in mice. (G) Acquisition of step-through passive avoidance during 3 successive testing trials in mice treated with or without EH-201. *p<0.05, **p<0.01 statistically significant compared with the SD group; ##p<0.01, statistically significant compared with the control groups by one-way ANOVA followed by Tukey's multiple comparison test.

FIG. 18 shows EH-201 induction of cellular EPO expression level in mice RPE cells. C57mice RPE cells were incubated with 0.4, 2, 10 μg/ml EH-201 in DMEM supplemented with 10% FCS. The cultures were incubated at 37° C. for 24 hours. After incubation period, whole cell lysates were prepared with lysis buffer. Total cell lysates were prepared and subjected to western blot analysis to detect the level of endogenous EPO. GAPDH was used as a loading control. Bars represent mean ±SD (n=3 different experiments; **p<0.01,***p<0.001).

FIG. 19A to FIG. 19D show induction of autophagy by EH-201. Primary mice hepatocytes were treated with EH-201 at different doses (0.6, 2.5, 10 and 40 μg/ml), rapamycin (autophagy activator,Rm, 50 nM) or 3-methyladenine (3MA, 10 mM) for 24 hours (A and B). The primary mice hepatocyte cultures under starvation (sty) acted as autophagy activation control (A, B). These treated cells were stained with monodansylcadaverine (MDC) followed by fluorescent microscopy examination (scale bars: 50 mm); and the fluorescent intensity was measured in spectroflurometer (B). Western immunoblotting was performed with hepatocyte lysate to study the expression of autophagic marker proteins LC3 using LC3 antibody (C, D). Kidney slices treated with EH-201 for 18 hours were used to study the effects of EH-201 on autophagy induction; analysis of autophagy induction was done by analyzing western blot against LC3. Quantification of LC3-II/LC3-I was performed using the immunoreactive bands with ImageQuant imaging software (Amersham Biosciences). Data are expressed as mean±SEM. **P<0.01, *P<0.05 compared with control.

FIG. 20A and FIG. 20B show that EH-201 induced autophagic activation is through hepatocyte growth factor (HGF) induction. Hepatocytes were treated with EH-201 at different doses (0.6, 2.5, 10 and 40 mg/ml) for 24 hours. rhEPO represented recombinent human EPO; iiiiHGF represented recombinant murine heaptocyte growth factor and nEPO-ab represented neutralizing EPO abtibody

### DETAILED DESCRIPTION OF PREFERRED EMBODIMENTS

The following specific examples are used for illustrating the present invention. A person skilled in the art can easily conceive the other advantages and effects of the present invention. The present invention can also be implemented by different specific cases be enacted or application, the details of the instructions can also be based on different perspectives and applications in various modifications and changes do not depart from the spirit of the creation.

Erythropoietin is abbreviated as EPO in this specification and drawings.

### EXAMPLE 1

### Extraction, Isolation and Characterization of EH-201

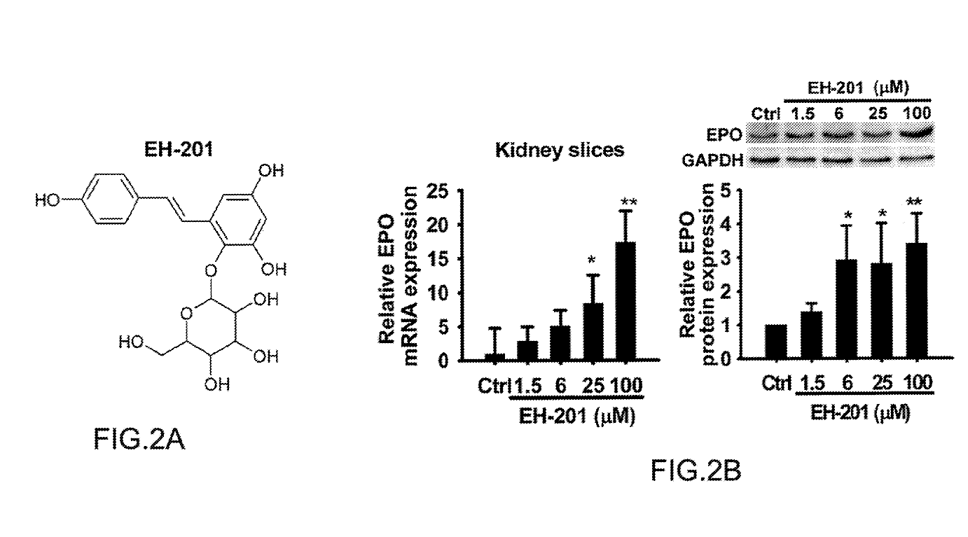

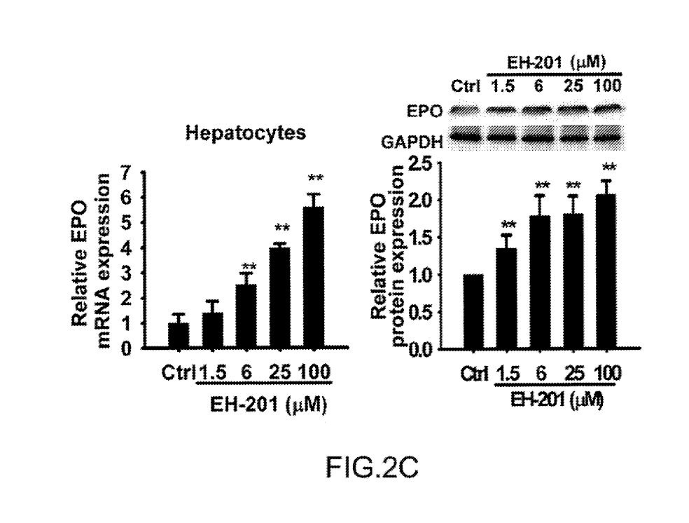

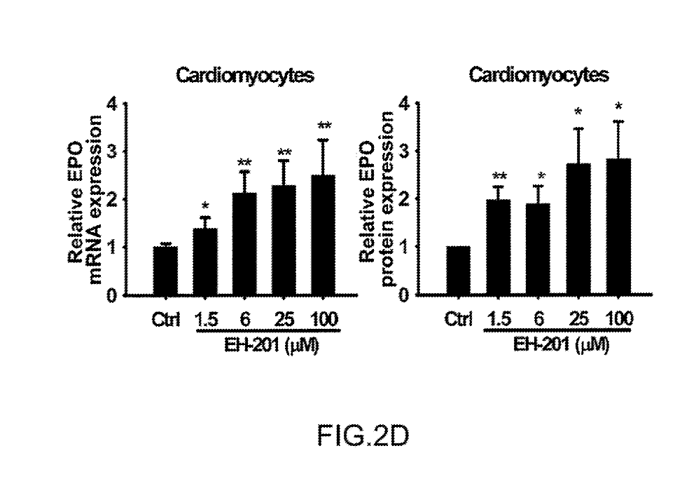

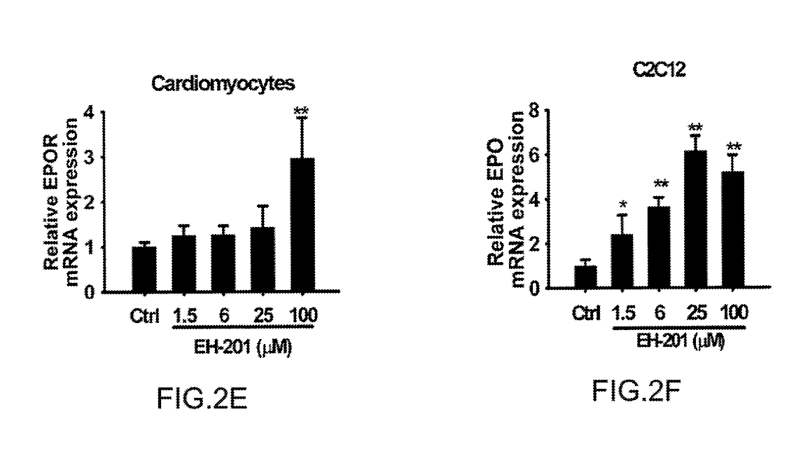

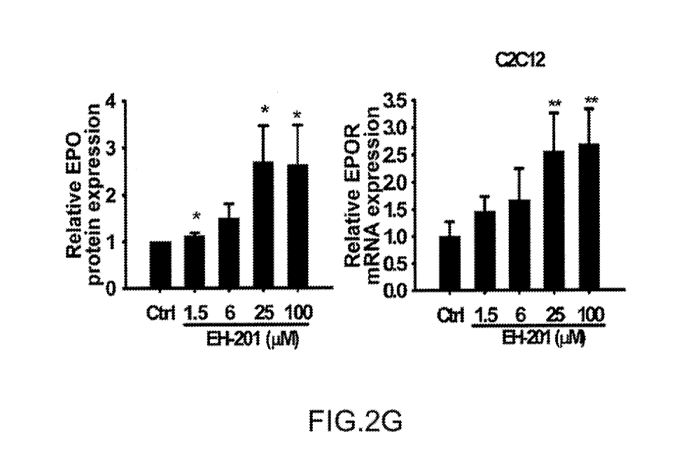

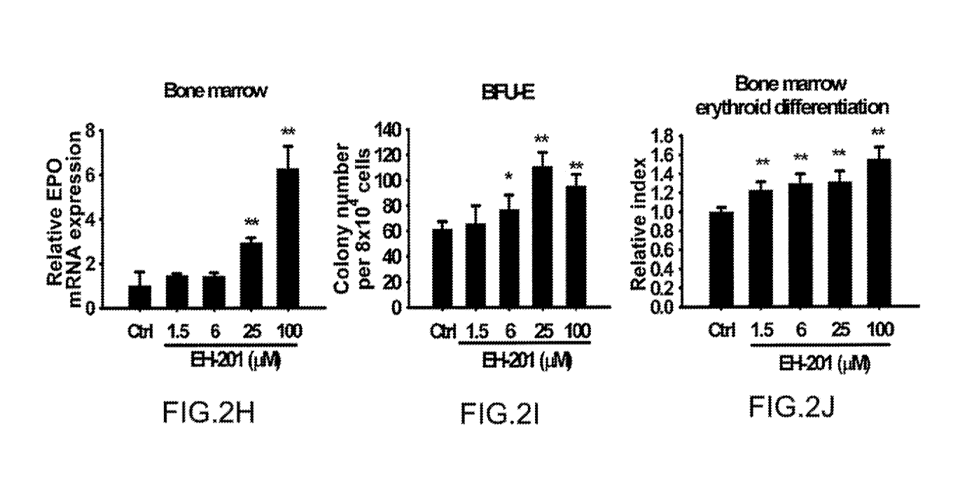

EH-201, 2,3,5,4′-tetrahydroxystilbene-2-o-beta-d-glucoside (hereinafter referred to as EH-201)(FIG. 2A) was extracted and purified to 99.2% purity. The dried and milled roots of Polygonum multiflorum Thunb. was extracted with 40% ethanol and then evaporated to form syrup. In order to enrich the target components, the extract was diluted twice with 15% ethanol, loaded on a Diaion HP-20 resin column and then eluted with sequential 20%, 40%, and 70% ethanol, respectively. The effluent of 40% ethanol was collected and evaporated. The 40% ethanol effluent was then redissolved in 10% ethanol by sonication and partitioned with ethyl acetate of equal volume five times successively. The residue of ethyl acetate was then passed through a Sephadex LH-20 column eluting with methanol. A pale yellow compound, EH-201, was obtained. The overall yield is about 0.5%o from the crude, dried, milled roots of Polygonum multiflorum Thunb. to final compound EH-201 in pure form (99.2%). For future clinical test purpose, the crystallization of this compound was further performed. The 30% aqueous-ethanolic solution of EH-201 was then placed into the −20° C. refrigerator overnight then placed into 4° C. refrigerator. An acicular crystal was obtained several days later.

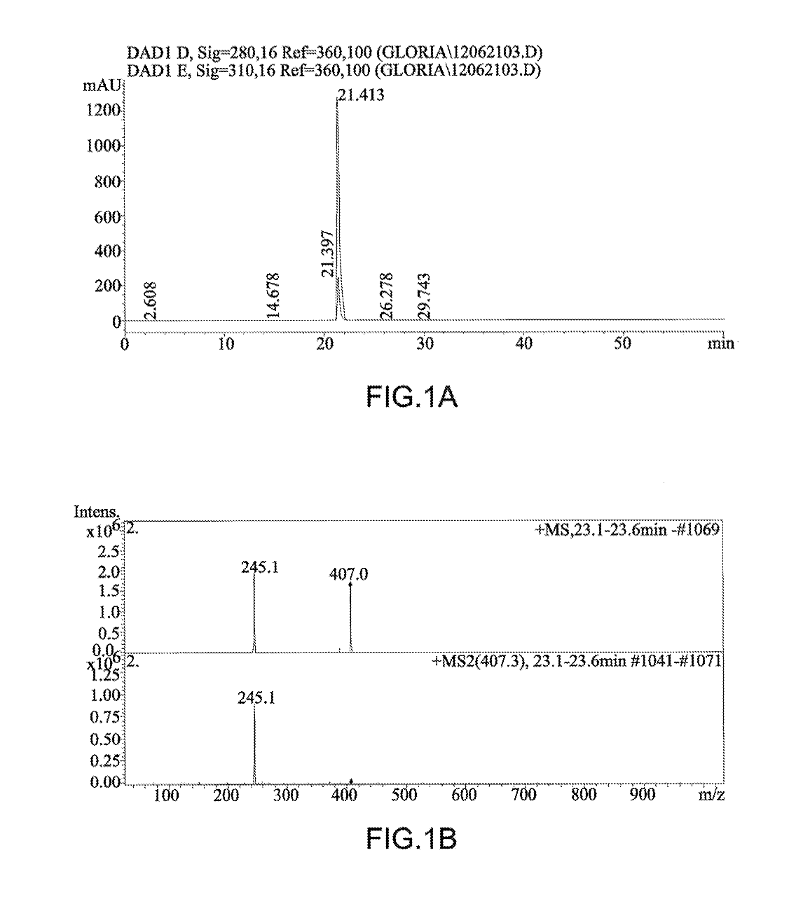

The chemical identity of EH-201 was confirmed by LC/MS/MS, UV, ¹H-NMR and proton-decoupled ¹³C-NMR data (FIG. 1 and Table 1), and ¹H-NMR and proton-decoupled ¹³C-NMR data sets using a Bruker NMR spectrometer. The proton and carbon chemical shifts of EH-201 are listed in Table 1. The LCMS data of the purified EH-201 was performed with a Bruker LC/MS/MS spectrometer Esquire 2000 in APCI (Atmospheric Pressure Chemical Ionization) mode with positive ion polarity, using a gradient of HPLC grade water and methanol over 60 minutes with a reverse phase C18 column (FIG. 1A). The LCMS data is exhibited in FIG. 1B showing the correct mass of EH-201 at m/z 407.0. The EH-201 ion at m/z 407.0 is further subjected to MS/MS analysis where only the 407.0 ion was isolated and fragmented. The resulting daughter ion at m/z 245.1 is consistent with the EH-201 loses its sugar moiety. Therefore, the compound was identified as 2,3,5,4′-tetrahydroxystilbene-2-o-beta-d-glucoside (TSG or THSG) (FIG. 2A).

TABLE 1
Proton (500 MHz) and carbon (125 MHz) chemical shifts* of EH-201

| Carbon | δₙ | δ_{c} |
|---|---|---|
| 1 | — | 133.8 |
| 2 | — | 138.0 |
| 3 | — | 152.2 |
| 4 | 6.57 (d, J = 2.75 Hz) | 103.7 |
| 5 | — | 156.1 |
| 6 | 6.21 (d, J = 2.75 Hz) | 102.8 |
| 1′ | — | 131.0 |
| 2′, 6′ | 7.41 (d, J = 8.6 Hz) | 129.4 |
| 3′, 5′ | 6.72 (dd, J = 8.6, 2.6 Hz) | 116.6 |
| 4′ | — | 158.5 |
| α | 7.67 (d, J = 16.5 Hz, trans) | 121.9 |
| β | 6.88 (d, J = 16.45 Hz, trans) | 130.2 |
| 1″ | 4.46 (d, J = 7.9 Hz) | 108.3 |
| 2″ | 3.23-3.75 (m) | 75.6 |
| 3″ | 3.23-3.75 (m) | 78.1 |
| 4″ | 3.23-3.75 (m) | 70.5 |
| 5″ | 3.23-3.75 (m) | 78.3 |
| 6″ | 333.23-3.76 (m) | 62.2 |

*All NMR spectra were recorded at 300 K and reference to the methanol solvent peak at 3.31 ppm for proton and 49.15 ppm for carbon resonances.

### EXAMPLE 2

### Activation of Mitochondrial Function and Haemoglobin Expression in Nonhaematopoietic Cells by the Compound of the Present Invention

This example describes various assays that are useful in evaluating the activation of mitochondrial function and haemoglobin expression in nonhaematopoietic cells by the compound of the present invention. The compound of the present invention is prepared according to the methods provided in Example 1. The potency of this compound is evaluated using a series of activity assays and these assays are further described in detail below.

### 1. Animals

Eight-to-ten-week-old specific pathogen-free C57BL/6J male mice (20-25 g), obtained from the National Laboratory Animal Centre (Taiwan) were housed 5-6 per cage at a constant temperature of 22±2° C. and fed standard laboratory chow (PMI, Brentwood, Mo., USA) and water ad libitum under a 12 hour dark/light cycle. The experimental protocol was approved by the Animal Research Committee of National Yang-Ming University (Guide for Animal Experiments, National Yang-Ming University). All efforts were made to minimize animal suffering, to reduce the number of animals used and to utilize alternatives to in vivo techniques, if available. All studies involving animals were reported in accordance with the ARRIVE guidelines for reporting experiments involving animals.

### 2. Cell Culture and Treatment

The C2C12 myoblast, HEK293, and TF-1 cells were purchased from Bioresources Collection and Research Centre (BCRC, Hsinchu, Taiwan). The C2C12 myoblasts were differentiated to myotubes and were treated with drugs for 24 hours. Ex vivo 250 pm-thick kidney slices were prepared from eight-to-ten-week-old C57BL/6J mice as previously described. The slices were treated with drugs in the gassed media (DMEM/F12 buffered with 15 mM HEPES and 20 mM sodium bicarbonate) in an atmospheric chamber at 37° C. with 50% O₂: 5% CO₂: 45% N₂ for 18 hours. Mouse primary hepatocytes were isolated and purified from eight-to-ten-week-old C57BL/6J mice as previously described and plated onto 1% gelatin-coated microplates in DMEM supplemented with 10% FBS (Gibco, Germany). After the hepatocytes had attached, fresh medium containing drugs was added for 24 hours. Neonatal C57BL/6J mouse cardiomyocyte cultures were prepared from post-natal one day-old C57BL/6J mice obtained from the Animal Centre at the National Yang-Ming University as described previously, and the isolated ventricular cells were resuspended in 10% FCS-containing M199 medium (Gibco, Germany). The cardiomyocytes were incubated in a humidified atmosphere at 37° C. with 5% CO₂ on plates precoated with 1% gelatin. The subconfluence of spontaneously beating cells was achieved after 48 hours of culture, after which treatments with various drugs were performed for 24 hours. The bone marrow progenitor cell cultures for the colony-forming assay and the haemoglobin colorimetric assay were prepared as previously described. In the knockdown experiment, the C2C12 myotubes were transfected with scrambled or PGC-1α-specific siRNA (Table 2) using the Lipofectamine 2000 reagent, according to the manufacturer's instructions (Invitrogen). These cultured cells were treated with rhEPO (recombinant human erythropoietin, Roche, Germany) or EH-201 or were co-incubated with EPO neutralizing antibody (R&D, MN) for the indicated time periods. Thereafter, the drug treated cell and tissue lysates were collected and homogenized to examine the specific expression of mRNA and protein, as well as their mitochondrial activity.

TABLE 2
Sequences of specific gene
used for Q-PCR primers, siRNA, and HRE

| Name | Sequence |
|---|---|
| GAPDH | FW: 5′-TGGCATTGTGGAAGGGCTCA-3′ |
| | REV: 5′-GGAAGAGTGGGAGTTGCTGT-3′ |
| EPO | FW: 5′-AATGGAGGTGGAAGAACAGG-3′ |
| | REV: 5′-ACCCGAAGCAGTGAAGTGA-3′ |
| EPOR | FW: 5′-TCTGGGAGGAAGCGGCGAGCT-3′ |
| | REV: 5′-GAGGAGAACCGGACGCCTCCGT-3′ |
| PGC-lα | FW: 5′-CGCCTTCTTGCTCTTCCTTT-3′ |
| | REV: 5′-TCTGCCTCTCTCTCTGTTTGG-3′ |
| Hb-α | FW: 5′-ATGTTTGCTAGCTTCCCCACCACCAAG-3′ |
| | REV: 5′-GGTGGCTAGCCAAGGTCACCAGCA-3′ |
| Hb-β | FW: 5′-TGATGCTGAGAAGGCTGCTGTCTCTG-3′ |
| | REV: 5′-GTGCCCTTGAGGCTGTCCAAGTGA-3′ |
| 16SiRNA | FW: 5′-CCGCAAGGGAAAGATGAAAGAC-3′ |
| (mtDNA) | REV: 5′TCGTTTGGTTTCGGGGTTTC-3′ |
| HK2 | FW: 5′-GCCAGCCTCTCCTGATTTTAGTGT-3′ |
| (mDNA | REV: 5′-GGGAACACAAAAGACCTCTTCTGG-3′ |
| control) | |
| VEGF | FW: 5′-GCAAGAGAGCGGGCTGCCTCGCAG-3′ |
| | REV: 5′-ACTTGATCACTTCATGGGACTTCT-3′ |
| PGC-lα siRNA | Sense: 5′-CAAAUGAGGGCAAUCCGUCUU-3′ |
| | Anti-sense: 5′-CAAAUGAGGGCAAUCCG |
| | UCUU-3′ |
| HRE | 5′-CCCTACGTGCTGTCCCTACGTGCTGTCCCTA |
| | CGTGCTGTCCCACGTGCTGT-3′ |

FW. forward:
REV. reverse:
HRE. hypoxia response elements.

### 3. Real-time PCR

The total RNA was extracted using the TRIzol reagent (Invitrogen) and was reverse transcribed by M-MLV Reverse Transcriptase (Promega). The EPO, EPOR, PGC-1α, Hb-α, Hb-β, and GAPDH mRNA expression were quantified by quantitative real-time PCR (Q-PCR) with an ABI 7500 sequence detector (Applied Biosystems) using SYBR Green Master MixR (ABI-7500).

The relative mRNA expression levels were determined using the TTCt method, with GAPDH as the endogenous control. The primers used are listed in Table 2.

4. Western blot

The total protein (50 μg) was separated by 12% SDS-PAGE, transferred onto PVDF membranes, and probed with antibodies against EPO, PGC-1α, GAPDH, PCNA (from Santa Cruz, Calif.), Sirt1 (Millipore, Billerica), or Hif-2α (Novus Biologicals, Littleton). Following incubation with the appropriate horseradish peroxidase-conjugated secondary antibody, the signals were visualized by ECL detection, according to the manufacturer's protocol (Perkin-Elmer).

5. Quantification of the mtDNA Copy Number

The total cellular DNA was purified using a conventional phenol-chloroform method, and the mtDNA copy number was measured, as previously described.

### 6. The MitoTracker Assay

The mitochondrial content was assessed by the MitoTracker microplate assay. The treated cells were loaded with 0.1 μM green fluorescent MitoTracker-Green (MTG, Invitrogen) for 60 minutes at 37° C. The intracellular MTG content was measured by fluorescence photometry (Thermo Scientific Inc.). Subsequently, the fixed cells were labeled with H33342 to assess the cell density. The MTG/H33258 fluorescence ratios were calculated.

### 7. Measurement of Citrate Synthase Activity

The citrate synthase activity was measured in tissue lysates. The changes in absorbance at 412 nm were measured, and the activity was expressed as μmol/min/mg protein.

### 8. TF-1 Cell Proliferation Assay

Cells of the tEPO-sensitive cell line TF-1 were seeded in 96-well microplates at a cell density of 1×10⁵ cells/ml in RPMI 1640 medium with 2% FBS, and the cells were treated with rhEPO and EH-201 with or without EPOR neutralizing antibody (Santa Cruz) for 48 hours. The cell numbers were determined by a trypan blue dye exclusion assay.

### 9. Rotarod Endurance Assessment

Before being divided into treatment groups, eight-to-ten-week-old C57B1/6J male mice were trained on a rotarod apparatus (14 rpm) for a maximum of 10 minutes for each of 3 consecutive training sessions per day for 3 days, and the animals that did not master this task were excluded from the experiments. After training, the qualified mice were randomly divided into EH-201-treating groups (10, 30 or 90 mg/kg per day, n=5 for each group) for seven days. On the testing day, each mouse was subjected to three trials on the rotarod at 22 rpm under a normoxic or hypoxic (8% O₂) atmosphere.

The endurance performance was measured over time until the mice suffered from exhaustion and fell off of the rotarod. The maximum trial length was 60 minutes, and there was a 30-minute rest period between each trial.

### 10. EPO ELISA

The serum EPO concentrations were analyzed using an ELISA kit specific for mouse EPO (R&D, MN), according to the manufacturer's instructions.

### 11. Doxorubicin-Induced Cardiomyopathy

Cardiomyopathy was induced in eight-to-ten-week-old C57B1/6J male mice by a single intraperitoneal (i.p.) injection of 15 mg/kg doxorubicin-HCl (Sigma-Aldrich), and the normal group was injected with saline (n=6). Seven days after the injection, the presence of doxorubicin-induced cardiomyopathy was confirmed with electrocardiogram by observing a prolonged S-T interval. An average eighty percent of injected mice were successful induced (27/34), and the ineffective mice were excluded from the EH-201 treating experiments. The cardiomyopathic mice were randomly divided into 4 cohorts comprising the control (Dox, n=9) and three EH-201-treating groups (n=6 for each group) for an additional week. EH-201 was administered orally by mixing it into the feed. The Dox group was fed a normal diet and EH-201-treating groups were fed normal diet containing different doses of EH-201 (10, 30 or 90 mg/kg per day). One week later, the mice were subjected to the rotarod endurance test, echocardiography and electrocardiogram. The mice were killed after electrocardiogram, and the isolated hearts were subjected to histological examination and haemoglobin analysis.

### 12. Haemoglobin Staining

The staining for haemoglobin in the isolated myocardium tissue lysates was performed with tetramethylbenzidine (TMBZ, Sigma-Aldrich), following nonreducing SDS-PAGE. The photography and scanning of the gels was performed using a Typhoon Trio™ imager (GE Healthcare). The TMBZ stain was removed from the gels by the addition of a 70 mM sodium sulfite solution. Thereafter, 30% isopropanol was used to replace the sodium sulfite, and then the gels were stained with Coomassie blue for analysis of the protein loading control.

### 13. Echocardiography and Electrocardiogram

The mice from all treatment groups were anaesthetized with isoflurane (0.75-1.5% inhalation), and echocardiographic measurements were taken in M-mode in triplicates for each mouse using an ATL HDI 5000 ultrasound system (Philips Medical Systems). To assess the electrocardiogram (ECG) parameters, three electrodes were utilized. The ECG tracings from lead I were recorded by means of an electrocardiograph connected to subcutaneous needle electrodes in the isoflurane-anaesthetized mouse. All probes were connected to an amplifier and digital converter for signal recording at the 100-mV range with low-pass 1 kHz and high-pass 1 kHz filters. An acquisition data system with LabVIEW software (National instruments, Inc.) was used to record and analyze the ECG signals.

### 14. Cisplatin-Induced Nephropathy

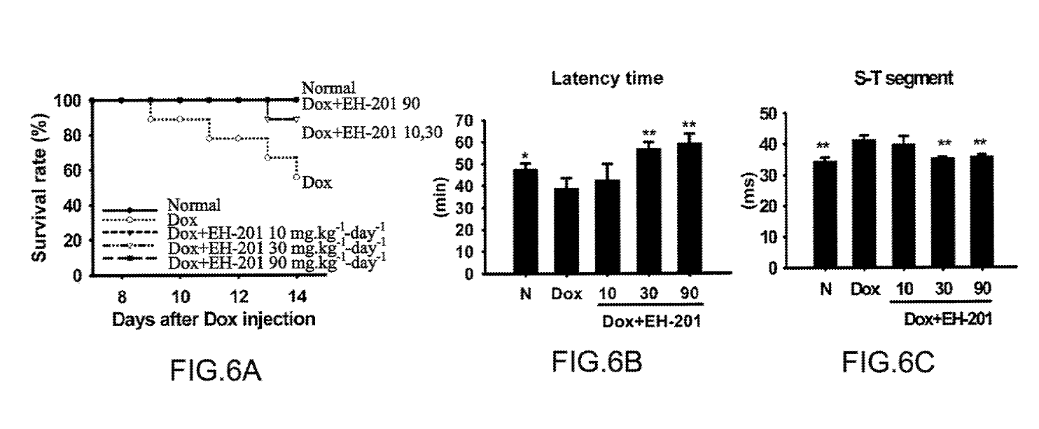

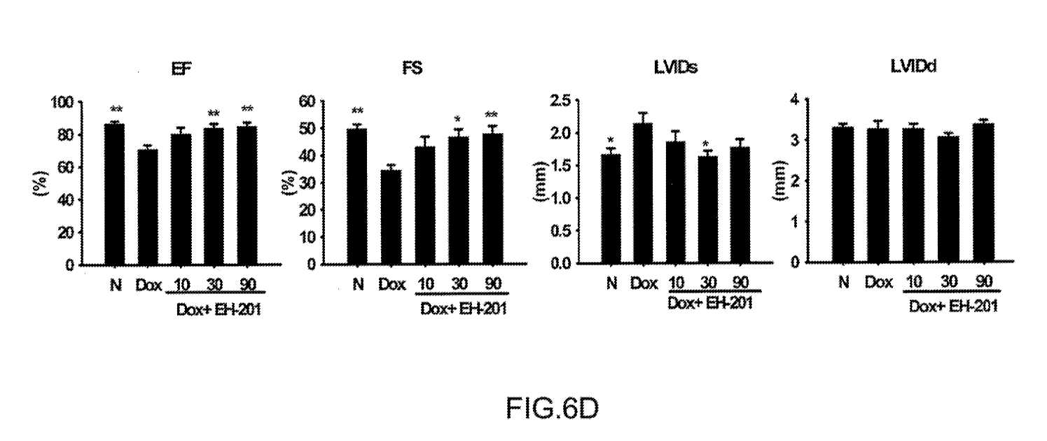

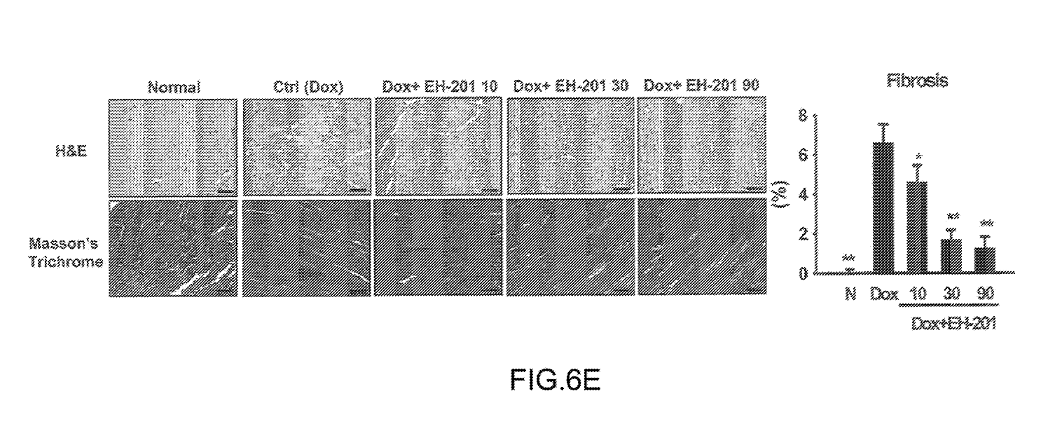

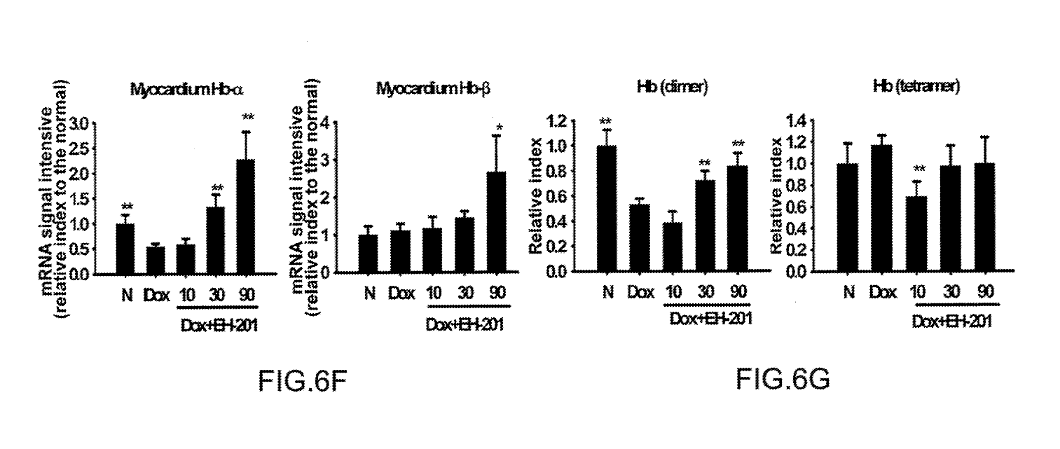

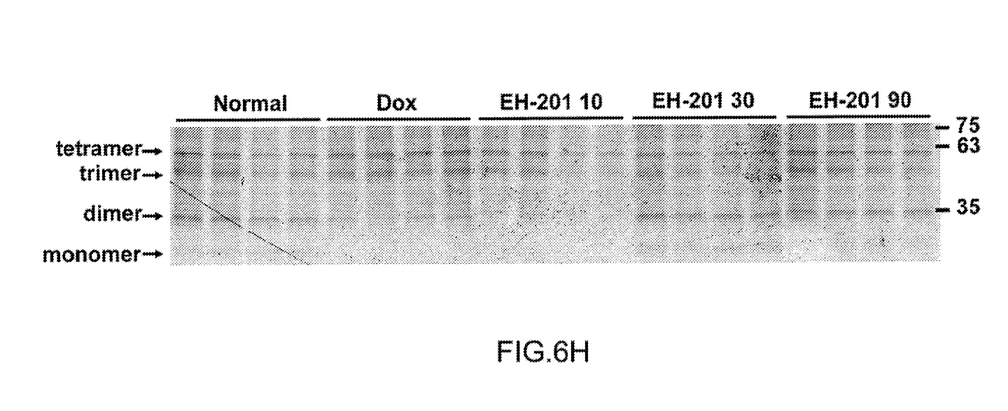

Forty eight-to-ten-week-old C57B1/6J male mice were i.p. injected with three doses of cisplatin (Sigma-Aldrich), following the scheme of 7, 6, and 6 mg/kg body weight, at 4-day intervals, and the normal group (n=6) was injected with saline (FIG. 6A). On day 13, the collected serum samples were assayed for the urea nitrogen content (BUN). Mice with BUN values greater than 100 mg/ dL were chosen for the experiment. An average seventy percent of injected mice were successful induced renal dysfunction (26/40), and the ineffective mice were excluded from the EH-201 treating experiments. The mice were subsequently divided randomly into 4 cohorts comprising the control (Ctrl, n=8) and three EH-201-treated groups (n=6 for each group) for an additional 2 weeks. Blood samples from all the mice were collected every 5 days. The RBC numbers were determined from the complete blood cell count using a Sysmex Kx-21 hematology analyzer (Sysmex America), and the serum BUN levels were determined through the urease GLDH method using a commercial kit (Urea FS, DiaSys, Germany).

### 15. Bone Marrow Progenitor Cell Colony-Forming Assay

The bone marrow cell suspensions were isolated and cultured from the femurs of six-week-old C57BL/6J male mice (National Laboratory Animal Centre, Taiwan) for assaying burst-forming units-erythroid (BFU-E). All cells were cultured in MEM-alpha medium containing 15% PBS (Gibco, Germany), 1% bovine serum albumin, 0.8% methylcellulose, 0.1 mM 2-mercaptoethanol (Sigma-Aldrich), 2 U/ml EPO (Roche, Germany), and 10 ng/ml IL-3 (Sigma-Aldrich). The colonies (≧50 cells) were counted on day 9 for BFU-E using an inverted microscope.

### 16. Haemoglobin Colorimetric Assay

For the detection of differentiated erythroid progenitors, the isolated bone marrow progenitor cells were cultured in the presence of the drug treatments in MEM-alpha medium containing 1% bovine serum albumin, 7.5 μM 2-mercaptoethanol, 1.4 mM L-glutamate (Sigma-Aldrich), 5 μM FeCl3 (Sigma-Aldrich), and 25 [mU/ml] EPO for 96 hours. Thereafter, the extracted haemoglobin was mixed with the 2,7-diaminofluorene (DAF, Sigma-Aldrich) working solution. The change in absorbance at 610 nm was continuously monitored at 25° C. for one minute. The initial rate of the reaction was measured, and the amount of Hb in the samples was determined from the Hb standard curve.

### 17. Luciferase Reporter Assay

HEK293 cells were transfected with a luciferase reporter plasmid (pGL3, Promega) containing four repeats of the minimal hypoxia response elements (HRE) from the EPO gene. The transfected cells were incubated with EH-201 under normoxia for 24 hours. The cells were kept under mimetic hypoxic (75 mM CoCl₂) or hypoxic conditions (5% O₂) as a positive control of Hif-1α activity. After the treatments, the cell lysates were harvested, and the luciferase expression was measured by the Dual-Luciferase Reporter Assay System (Promega).

### 18. Histological Analysis

The heart and kidney tissues were fixed with 10% formalin for paraffin embedding. Paraffin sections (cross-section for the heart) of 5 pm thickness were prepared for the H&E and Masson's trichrome staining protocols. For the analysis of myocardial fibrosis, 6 random photomicrographs were taken in the viable myocardium at a 400× magnification for each animal. The extent of fibrosis in these photomicrographs was quantified by a blinded observer using the ImageJ program from NIH.

19. Isolation Retinal Pigment Epithelial Cells Sheets from Mice and Cell Culture

Intact eyes were removed quickly from 6-8 week old C57/BL6 mice (National Laboratory Animal Center, Taiwan R.O.C.) and stored in ice cold PBS, which contained: 8.0 g/L NaCl, 0.2 g/L KCl, 0.8 μL KH₂PO₄, and 1.15 g/L NaH₂PO₄. Eyes were washed twice in growth medium (GM) consisting of Dulbecco's modified eagle's medium (DMEM) containing high glucose, 10% FBS, 1% penicillin/streptomycin, 2.5m ML-Glutamine and 1% non-essential amino acids. After washing, the eyes were then transferred into fresh PBS for dissection. Using microdissection scissors and an upright dissection microscope, a circular incision was made around the ora serrata of each eye. The posterior eyecup containing the neural retina and the lens were placed in fresh GM medium and incubated for 20 minutes at 37° C. in 5% CO₂ incubator to facilitate separation of the Retinal Pigment Epithelial (RPE) cell sheets from the neural retina. After removal of the RPE sheets from the neural retina, intact sheets of RPE cells were peeled and collected in an eppendorf tube. RPE cells were centrifuged at 1500 rpm for 5 minutes and resuspended in GM medium. The cell suspension (0.5 ml) was added to a 12-well plate. Cells were cultured at 37° C. in 5% CO₂ for 10 days, with a change of medium (GM) every other day. After 10 days the cells were washed with EDTA and then trypsinized for 4 minutes to detach the cells. The cells were collected in a tube, centrifuged at 1000 rpm for 5 minutes and resuspended in DMEM, 10% FBS, PEN/strep, 1-glutamine, sodium bicarbonate. The cells were plated in 6 cm dish until they reached confluence, at which time they were trypsinized and grown in a larger dish.

C57mice RPE cells were incubated with 0.4, 2, 10 μg/ml EH-201 in DMEM supplemented with 10% FCS. The cultures were incubated at 37° C. for 24 hours. After incubation period, whole cell lysates were prepared with lysis buffer. Total cell lysates were prepared and subjected to western blot analysis to detect the level of endogenous EPO. GAPDH was used as a loading control.

### 20. Statistics

All results are expressed as the mean±SEM. The statistical analysis was performed using Student's t-test. One-way ANOVA was used to examine the differences across the animal experimental groups. The posthoc differences between the means of the experimental groups were determined via Tukey's test. P<0.05 was considered significant.

### 20. Results

### (1) EH-201 is a Potent EPO Inducer

To determine whether EH-201 has the ability to induce EPO expression, kidney slices and hepatocytes were treated with EH-201 ex vivo. EH-201 was observed to dramatically induce EPO mRNA and protein expression in a concentration-dependent manner in the kidney slices and hepatocytes (FIGS. 2B and 2C). According to the gene expression pattern of EPO in human tissues in the publicly available database created by Su, A.I., et al., the EPO transcript is expressed at a surprisingly high level in human cardiomyocytes. Therefore, whether EH-201 can also induce EPO expression in neonatal mice cardiomyocytes and C2C12 myocytes was also tested. It was observed that EH-201 concentration-dependently induced the expression of EPO and EPO receptor (EPOR) in the primary cardiomyocytes and C2C12 myocytes (FIGS. 2D to 2G). Because the bone marrow progenitor cells can express EPO to mediate hematopoiesis, bone marrow cells were cultured with EH-201 to examine its effect on erythropoiesis. The expression of EPO mRNA was increased in the bone marrow cells exposed to EH-201 (FIG. 2H). EH-201 significantly increased the number of BFU-E colonies (FIG. 21) and Hb expression in a concentration-dependent manner (FIG. 2J). Accordingly, EH-201 is an EPO inducer.

### (2) The Induction of Mitochondrial Biogenesis by EH-201 is Mediated by EPO

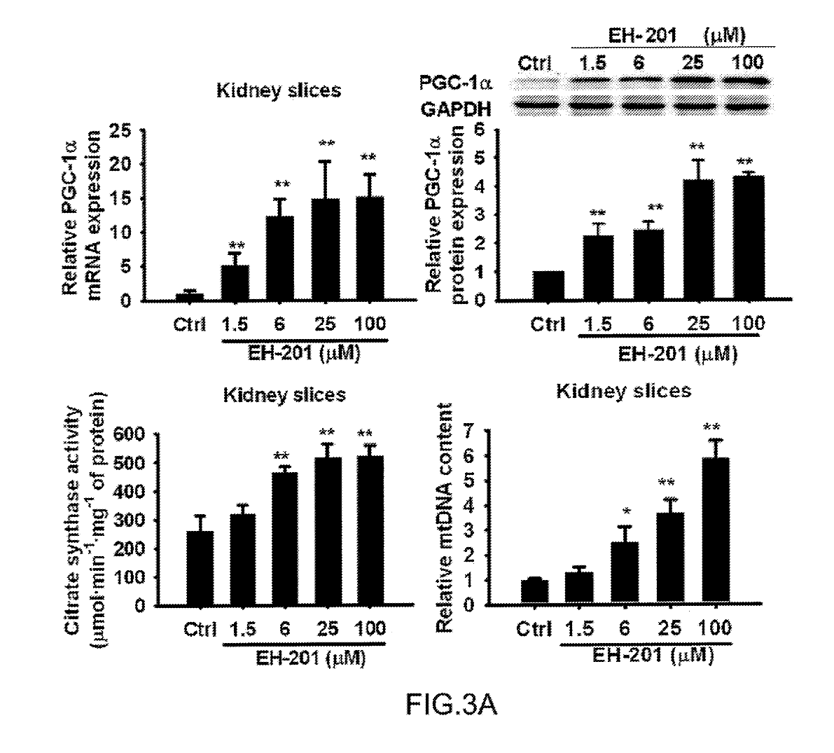

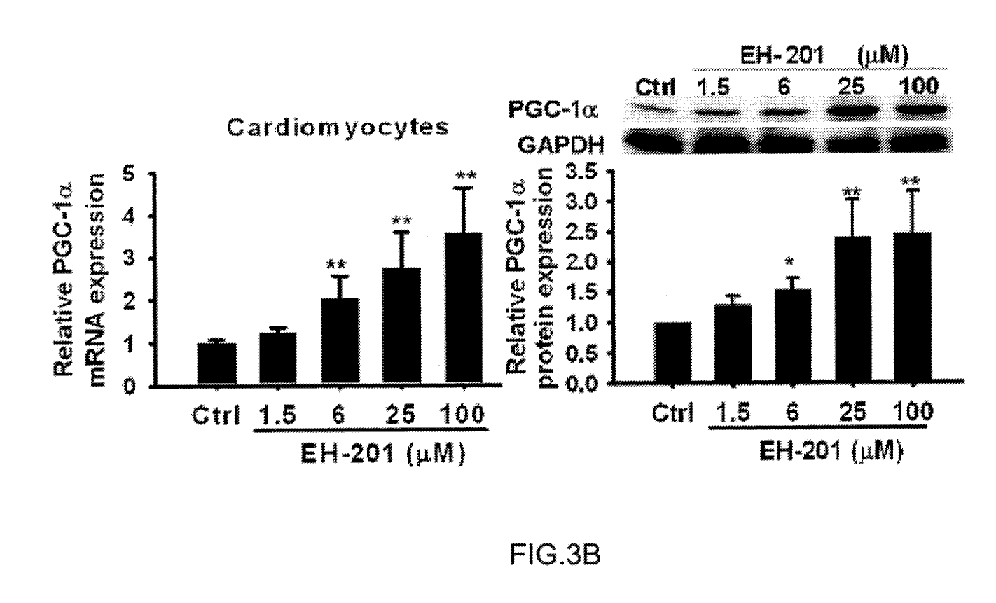

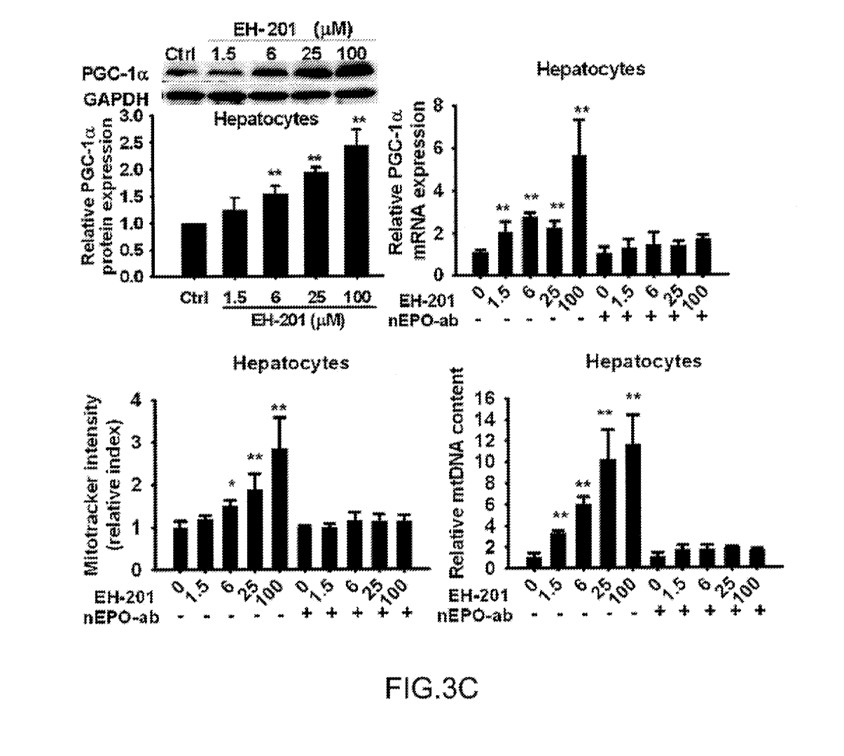

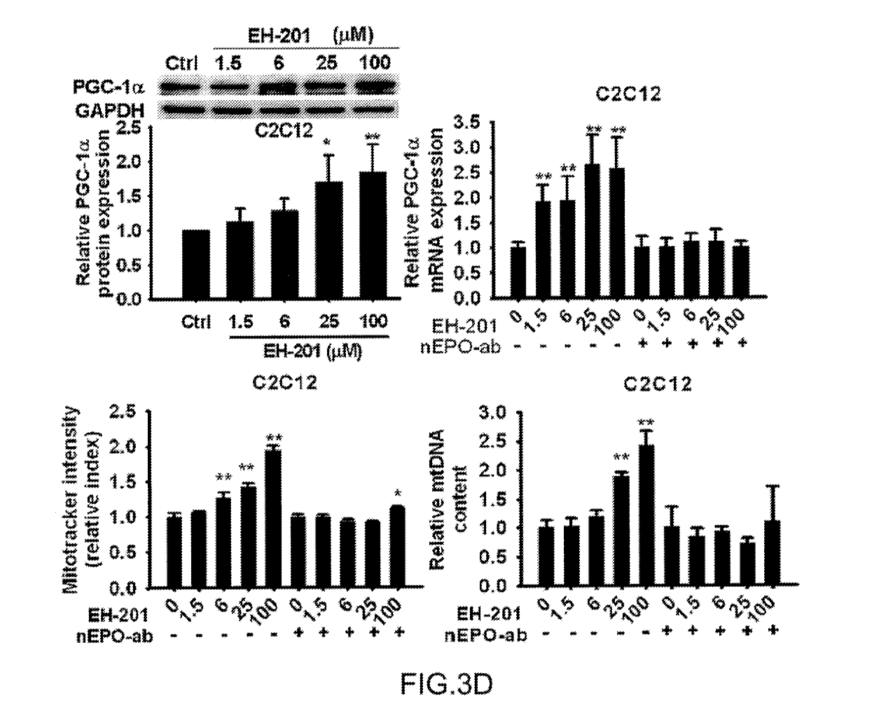

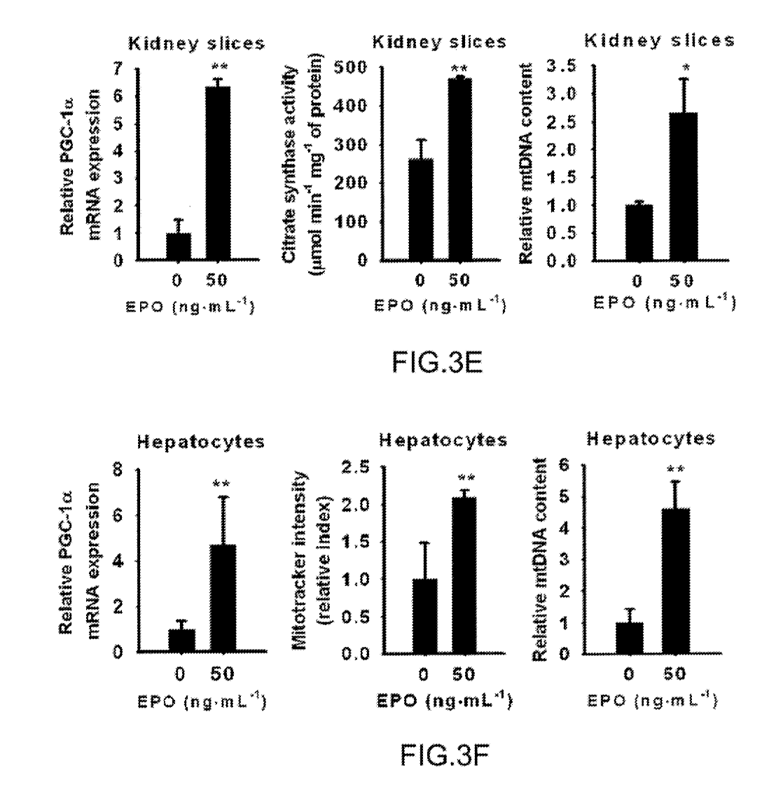

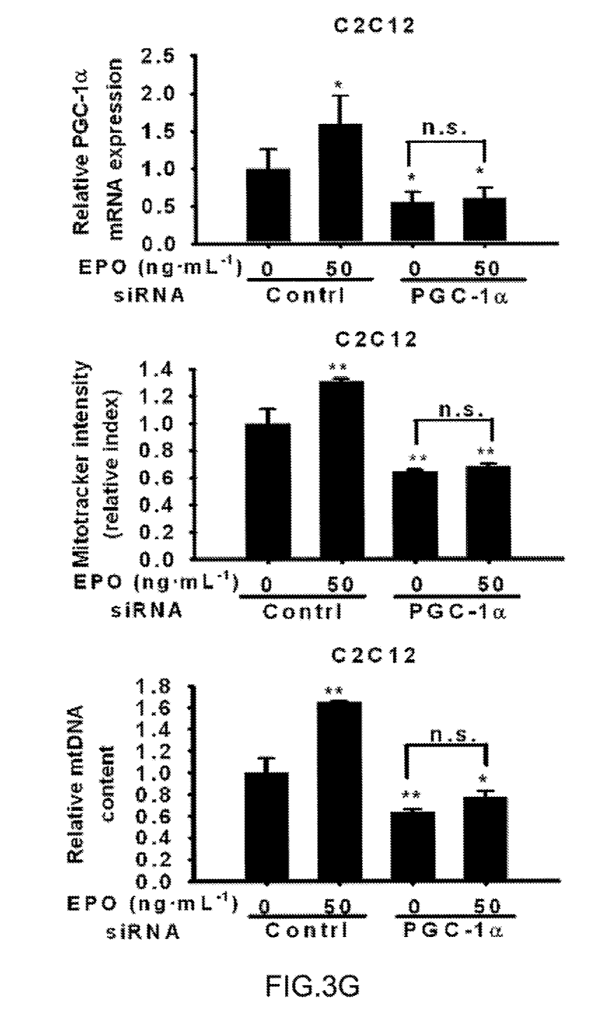

To determine whether EH-201 influences mitochondrial biogenesis, a series of experiments were performed to test the effects of the EPO inducer in nonhaematopoietic cells. In the EH-201-treated kidney slices, the activity of the mitochondrial marker enzyme citrate synthase increased in a concentration-dependent manner, and a dramatic increase in the mitochondrial copy number and PGC-1α expression was also observed (FIG. 3A). The stimulatory effects of EH-201 on mitochondrial biogenesis were also observed with hepatocytes, cardiomyocytes, and C2C12 myocytes (FIGS. 3B to 3D). However, neutralizing-EPO antibody treatment abolished the effects of EH-201-induced mitochondrial biogenesis (FIGS. 3C and 3D), whereas EPO treatment increased PGC-1α expression and mitochondrial biogenesis (FIGS. 3E to 3G). It was next examined whether these effects were mediated by a PGC-1α-dependent pathway using PGC-1α-specific siRNA-transfected C2C12 myocytes. The PGC-1α siRNA resulted in a 44% reduction in PGC-1α mRNA expression and a concomitant failure of EPO to induce mitochondrial biogenesis (FIG. 3G), which indicated that the activation of mitochondrial biogenesis by EPO is PGC-1α-dependent. Additionally, because the mammalian sirtuin (Sirtl) regulates mitochondrial function and biogenesis in the skeletal muscles and liver along with PGC-1α, Sirt1 expression was investigated and it was observed that EH-201 treatment increased Sirt1 expression (FIG. 8), which indicates that EH-201's induction of EPO-mediated mitochondrial activity might occur through the Sirt1/PGC-1α pathway. Therefore, the increase in PGC-1α due to EH-201 is dependent on the induction of mitochondrial biogenesis in nonhaematopoietic cells by increased EPO levels.

### (3) The Induction of Haemoglobin Expression in Nonhaematopoietic Cells by EH-201 is Mediated by EPO

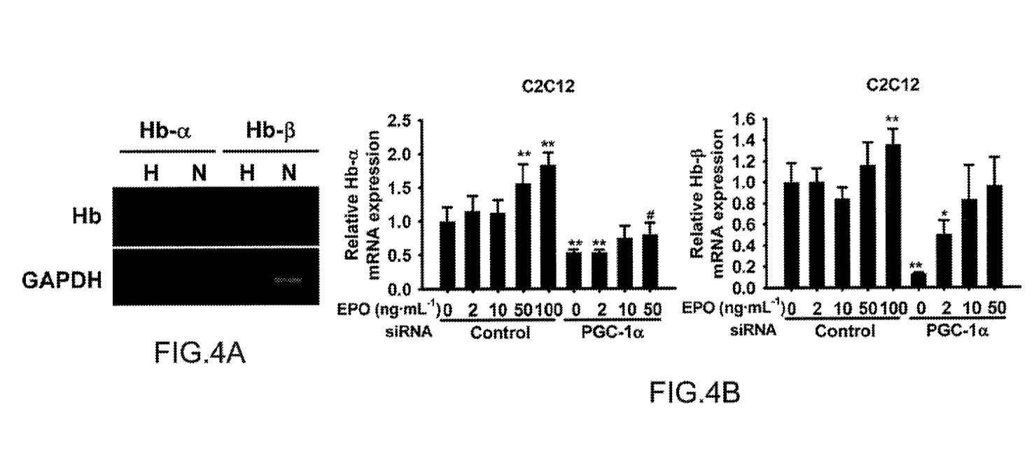

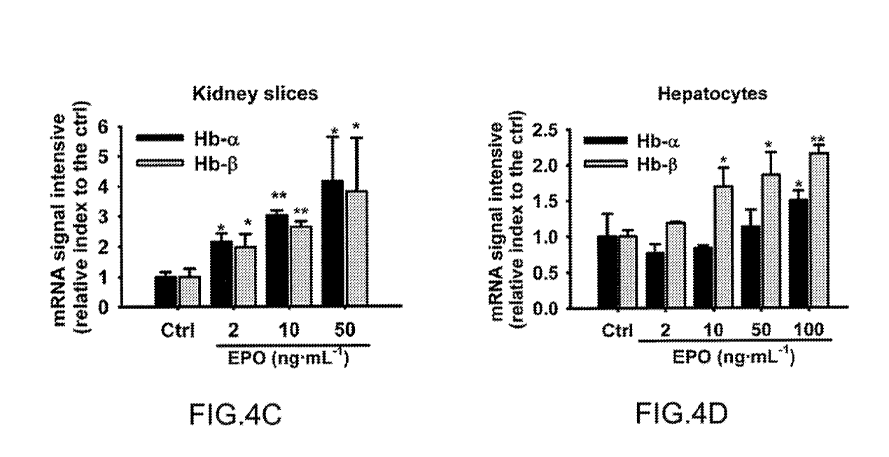

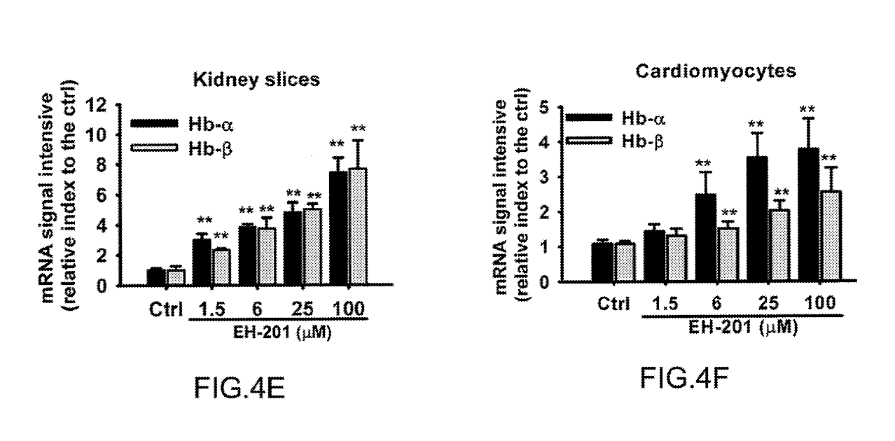

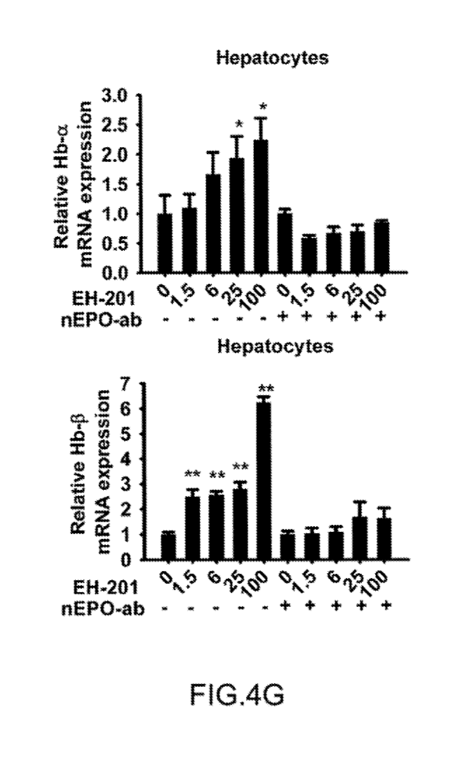

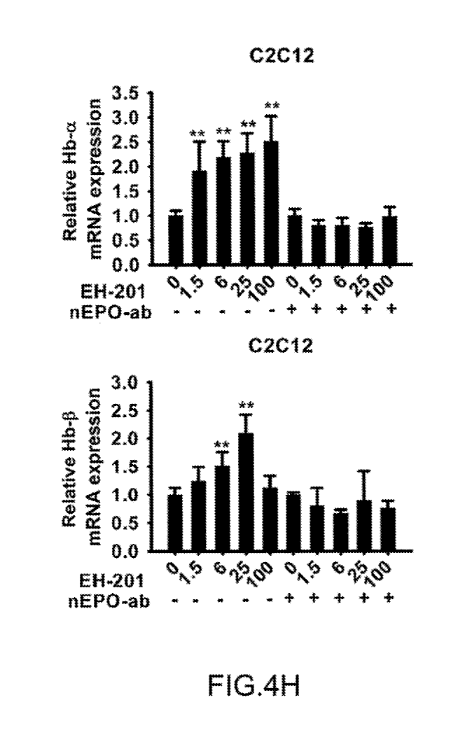

It was further determined whether the expression of haemoglobin (Hb) was regulated by hypoxia inducible EPO signaling in nonhaematopoietic cells. In vitro experiments were performed by incubating C2C12 cells in the absence and presence of hypoxic conditions. The exposure of the C2C12 myoblasts to hypoxia resulted in a noticeable increase in the expression of Hb-α and Hb-β (FIG. 4A). In the EPO-treated C2C12 myocytes, the induction of Hb-α expression was more susceptible to treatment than that of Hb-β (FIG. 4B). In addition, the expression of both Hb-α and Hb-β was increased in a concentration-dependent manner in the EPO-treated kidney slices, whereas only the expression of Hb-β was susceptible to induction in the EPO-treated hepatocytes (FIGS. 4C and 4D). The expression of Hb subunits was significantly increased in EH-201-treated nonhaematopoietic cells (FIGS. 4E to 4H), and this increase was abolished by concomitant neutralizing-EPO antibody treatment (FIGS. 4G and 4H). Studies were also conducted to determine the role of EPO signaling in the induction of Hb expression by PGC-1α siRNA. It was observed that the reduction of PGC-1α expression in C2C12 myocytes led to a decrease in the expression of both Hb-a and Hb-β mRNA and also resulted in a decrease in the inducing effect of EPO on Hb-a (FIG. 4B). Hence, the regulation of Hb expression in nonhaematopoietic cells occurs through both EPO mediated PGC-1α-dependent and PGC-1α-independent pathways. These results show that EPO-mediated signaling is required for EH-201's induction of haemoglobin expression in nonhaematopoietic cells.

### (4) EH-201 as an Enhancer of EPO to EPOR Binding Instead of Involving Hif-α Activation

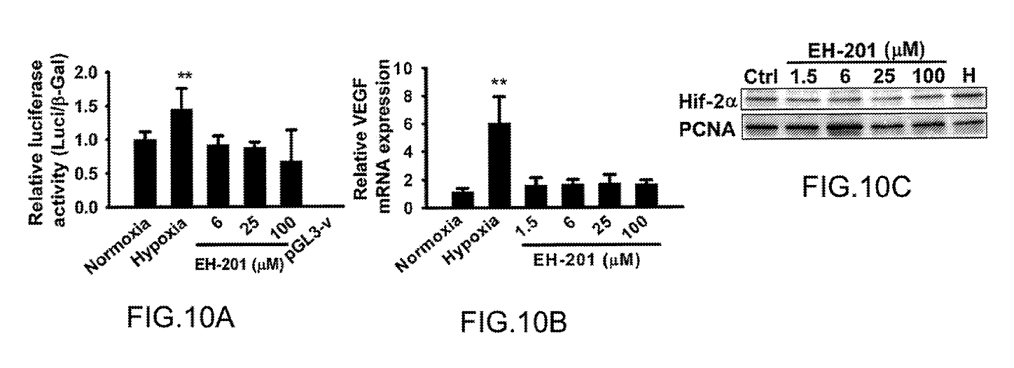

To examine the mechanism behind EH-201's activity, computational docking methods were carried out to predict the binding of EH-201 to EPOR. It was found that EH-201 binds preferentially to the EPO-bound EPOR complex (EPO/EPOR) rather than the EPO-free naive EPOR (estimated total intermolecular energy −7.48 kcal/mol and −6.30 kcal/mol, respectively). Autodock identified more than two preformed binding sites in the EPO/EPOR complex for EH-201 with negative favorable binding free energy, and the predicted interaction residues on EPOR (Met¹⁵⁰, Thr¹⁵¹, FIG. 9) involved the hot-spot residues located in loop 5. Because EPO autocrine activity also plays an important role in EPOR activation and the regulation of EPO production, the hypothesis that EH-201 may act as binding enhancer of EPO to EPOR, thus enhancing the EPOR activation was tested. A TF-1 cell (EPOR positive) proliferation assay was performed to address the EPO biological activity. It was observed that rhEPO induced the proliferation of TF-1 cells concentration-dependently, whereas, in the absence of rhEPO, EH-201 alone was unable to induce cell proliferation (FIG. 4I). In the presence of even very low concentrations of rhEPO, e.g., 2 ng/ml, EH-201 significantly induced TF-1 cell proliferation in a concentration dependent manner. The addition of neutralizing EPO or neutralizing EPOR antibodies both significantly reduced TF-1 cell proliferation (FIG. 4I). These data indicate that EPO is required for the activity of EH-201 and that EPO/EPOR complex may be the target of EH-201, which serves as an enhancer of EPO and EPOR binding. It was also investigated whether EH-201-induced expression of EPO involves the activation of the hypoxia-inducible factor (Hif), as EPO expression is regulated by Hif. As shown in FIG. 10A, using a hypoxia response element driven luciferase reporter to assess the activation of Hif-1α, EH-201 treatment did not activate the promoter activity. Furthermore, Hif-1α targeted vascular endothelial growth factor (VEGF) expression was upregulated during hypoxia, whereas EH-201 did not alter the VEGF expression (FIG. 10B). EH-201 treatment also did not stabilize the Hif-2α protein levels (FIG. 10C). These findings indicate that the induction of EPO by EH-201 is not due to the activation of Hif-1α or Hif-2α.

### (5) EH-201 Administration Enhances the Endurance Performance of Mice

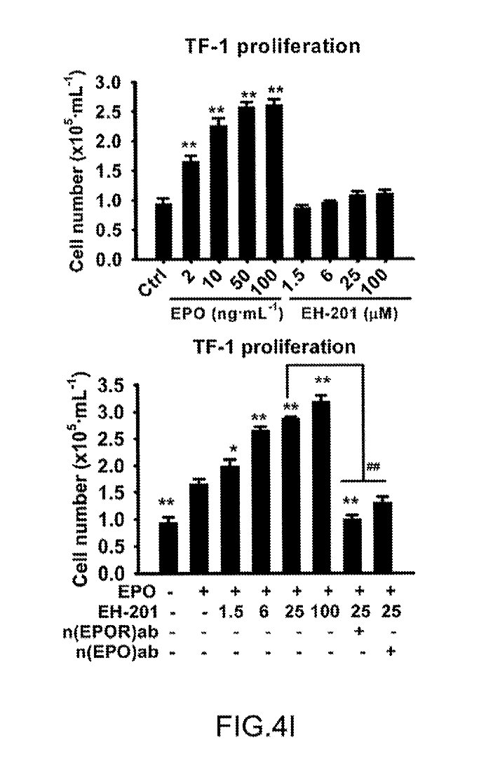

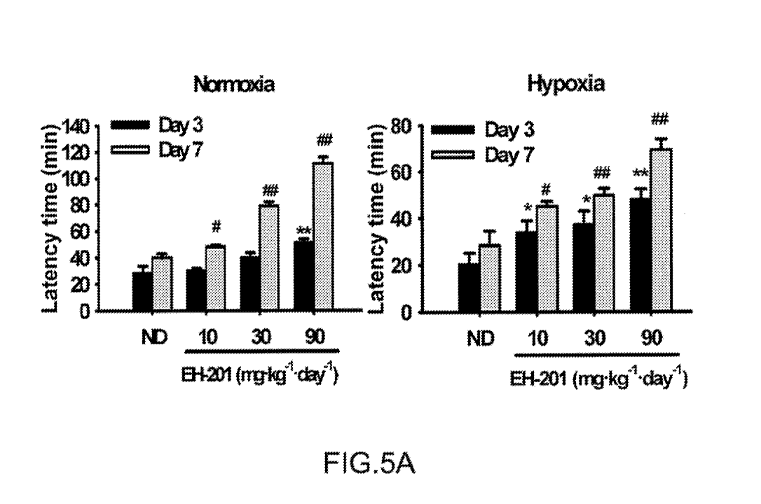

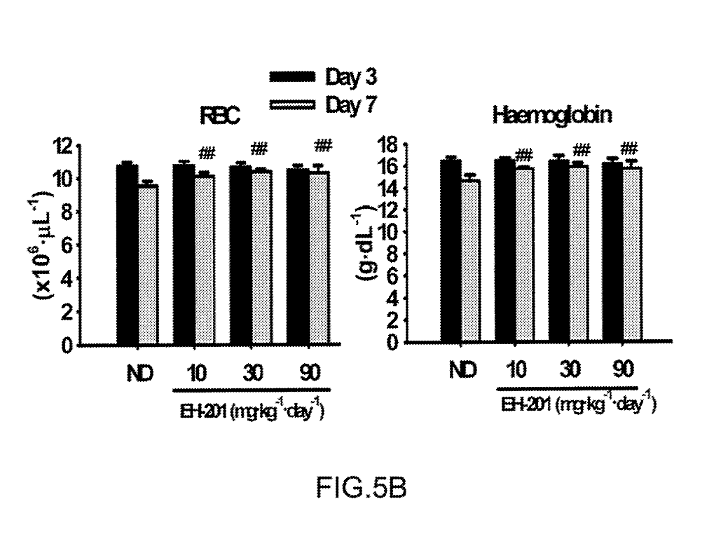

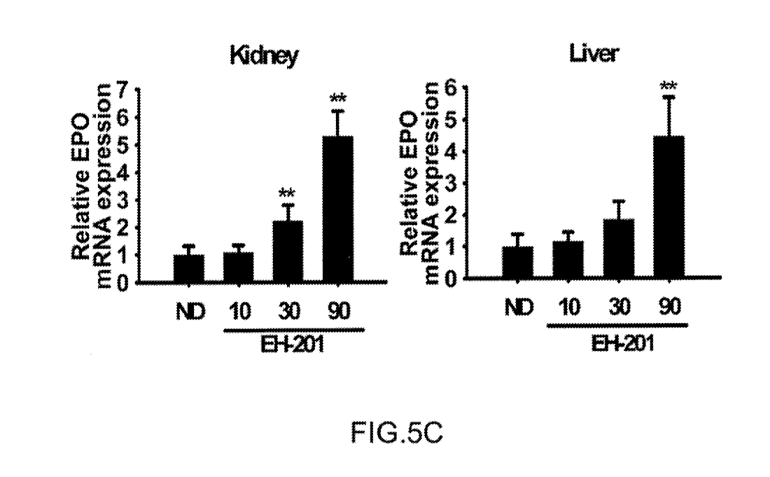

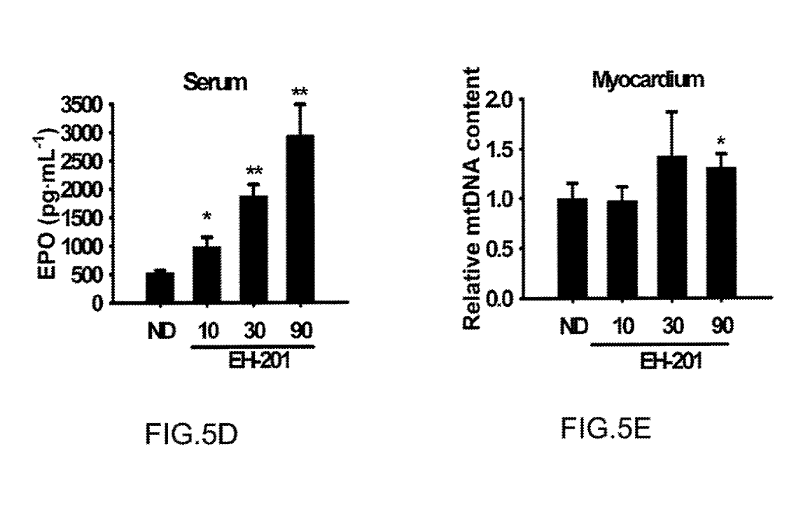

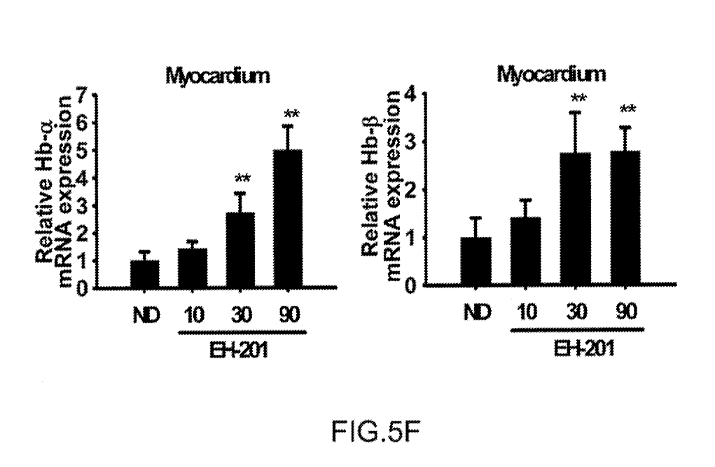

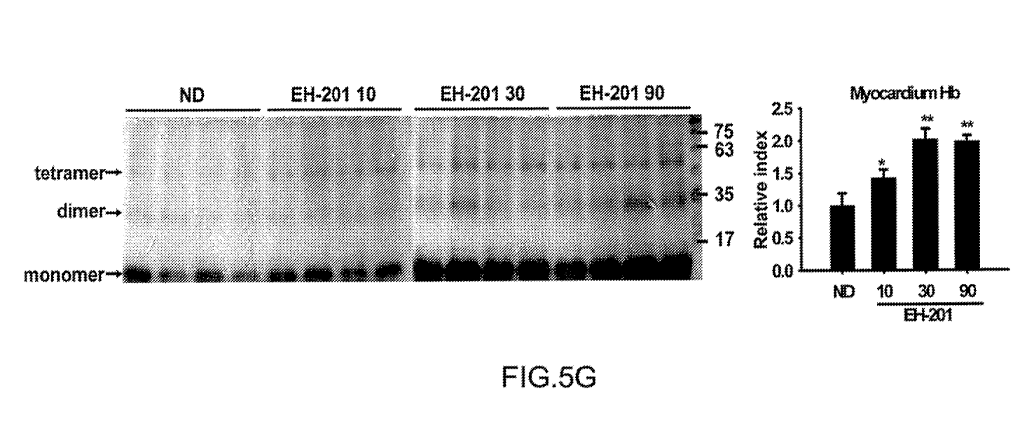

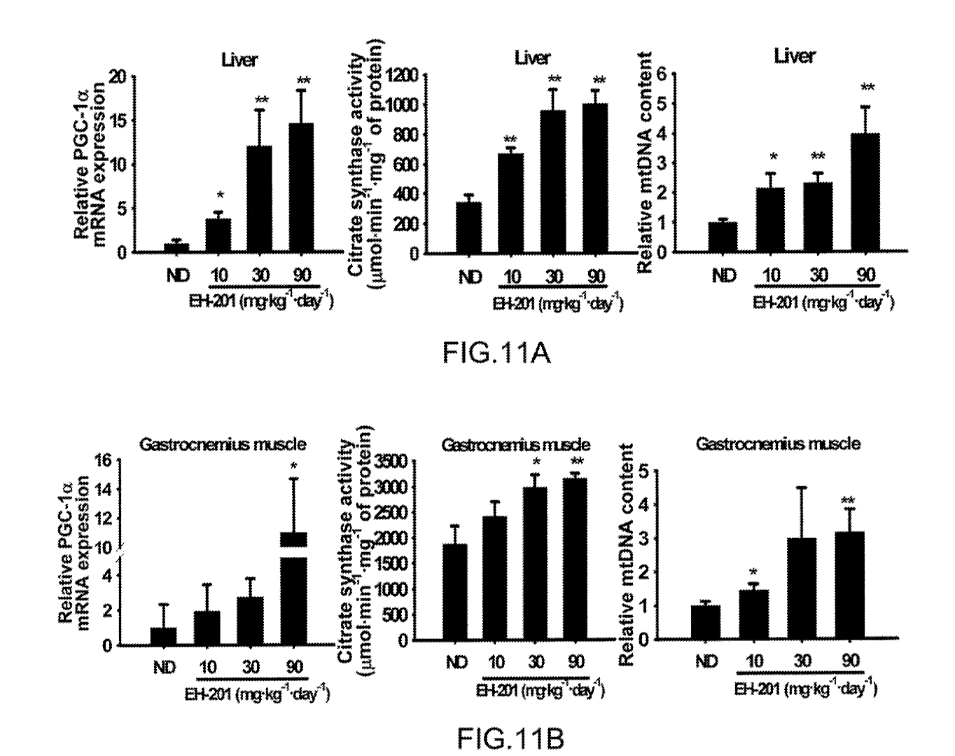

Given EH-201's EPO-inducing effect, whether EH-201 could enhance endurance performance in mice undergoing hypoxic stress was tested. Notably, the administration of EH-201 for 3 days increased the run time to exhaustion under both normoxia and hypoxia in a dose-dependent manner (FIG. 5A), with a further enhancement at 7 days. However, there was only a slight increase in RBC counts and Hb content in the peripheral blood (FIG. 5B), which indicated that EH-201 increased the RBC numbers by inducing an increase in the endogenous EPO levels (FIG. 5D), as confirmed by the induction of the production of renal and hepatic EPO (FIG. 5C). The expression of Hb-a and Hb-β in the myocardium of the EH-201-treated mice was significantly increased (FIG. 5F), as confirmed by an increase in Hb protein expression observed with TMBZ staining (FIG. 5G). High doses of EH-201 also induced cardiac mitochondrial biogenesis (FIG. 5E). Furtheii lore, EH-201 treatment resulted in significantly increased PGC-1α expression and mitochondria content and activity in the liver and skeletal muscles (FIGS. 11A and 11B). These results show that EH-201 treatment dramatically enhances the endurance performance and hypoxic tolerance of the mice via the induction of increased endogenous EPO expression and the stimulation of mitochondrial biogenesis and Hb expression in nonhaematopoietic tissues.

### (6) Therapeutic Effect of EH-201 on Established Doxorubicin-Induced Cardiomyopathy

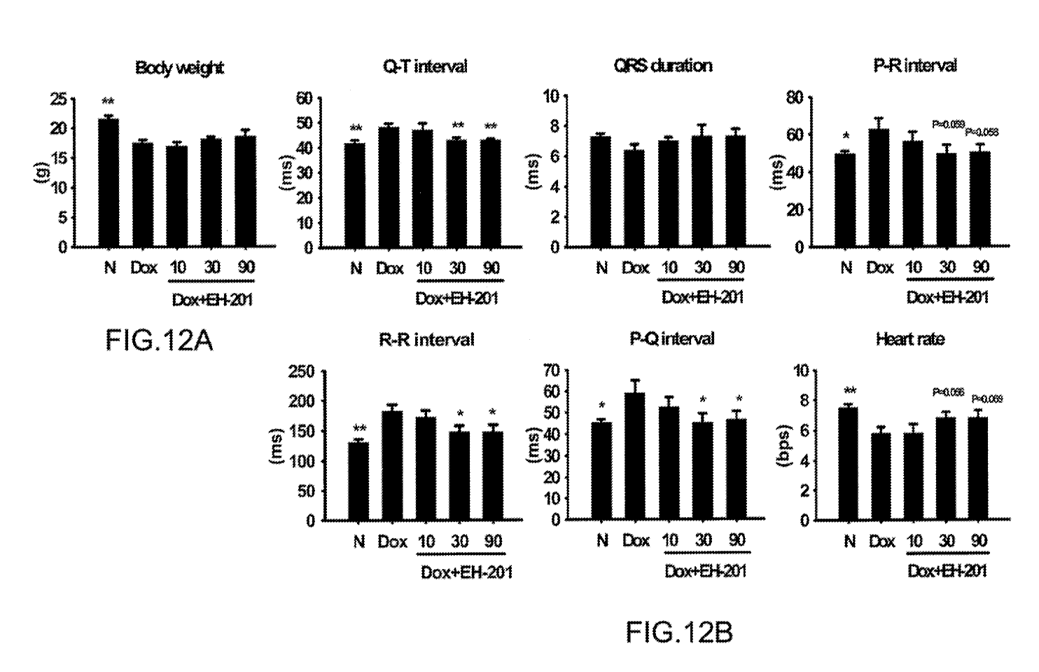

To assess the therapeutic potential of EH-201 in myocardial ischemia, a doxorubicin (Dox)-induced cardiomyopathy model was used. One week after Dox injection, the cardiomyopathic mice, as identified by ECG measurements, were started on EH-201 treatment for seven days to examine EH-201's therapeutic effects. The survival rates of the EH-201-treated groups were seen to improve, and the high-dose group remained alive until the end of the study period (FIG. 6A). Following the hypoxic rotarod endurance tests, although none of the groups recovered from the initial changes in body weight (FIG. 12A), the endurance performance activity of the EH-201-treated groups was found to be robustly increased (especially for the 30 and 90 mg/kg doses), whereas that of the Dox group was significantly reduced (FIG. 6B). Myocardium injury was measured by ECG up to 2 weeks following the injection of Dox, and these ECG parameters were significantly abnormal, which reflected the extensive cardiac damage caused by Dox (FIG. 6C). Seven days after the administration of EH-201, these ECG signs were significantly recovered in the mice treated with EH-201 (30, 90 mg/kg), which indicated an improvement in cardiac activity (FIGS. 6C and 12B). Echocardiography performed 2 weeks after Dox administration demonstrated that mice receiving Dox alone had significant cardiac functional deterioration, as characterized by decreased ejection fractions and fractional shortening. Mice receiving EH-201 (30, 90 mg/kg) treatment had significantly greater ejection fractions and fractional shortening, by comparison (FIG. 6D). However, there were no significant differences in the left ventricular diameters at the systole and diastole between the groups. There results indicate that treatment with EH-201 significantly mitigated the Dox-induced impairment of cardiac function. In addition, the Dox-damaged hearts presented with cytoplasmic vacuolization, myofibrillar loss, and developed myocardial fibrosis, which were ameliorated by EH-201 treatment (FIG. 6E). The image quantification results indicated that Dox increased the area of fibrosis in the ventricular endomysium, compared with normal mice (normal, 1.71±0.18% versus Dox, 8.31%±0.94%, (FIG. 6E), whereas fibrosis was almost absent in the mice treated with medium to high doses of EH-201. also It was also observed that Hb expression in the isolated myocardium of the EH-201-treated mice was upregulated (FIG. 6F, 30, 90 mg/kg) and Hb dimer forms increased (FIGS. 6G and 6H). Taken together, these data show that EH-201 has therapeutic effects, improving the cardiac function and ischemic tolerance of the Dox-induced cardiomyopathic mice.

### (7) EH-201 Ameliorates Anaemia and Renal Function in Cisplatin-Induced Nephropathy

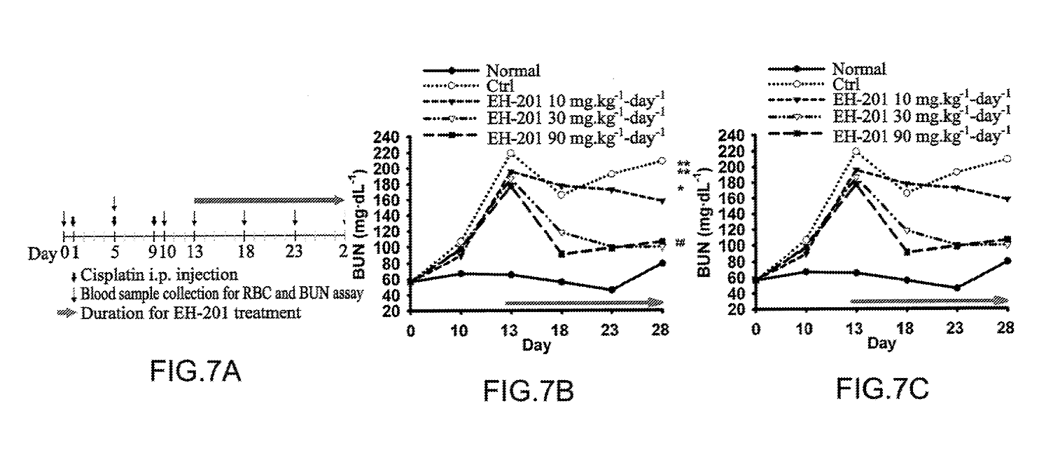

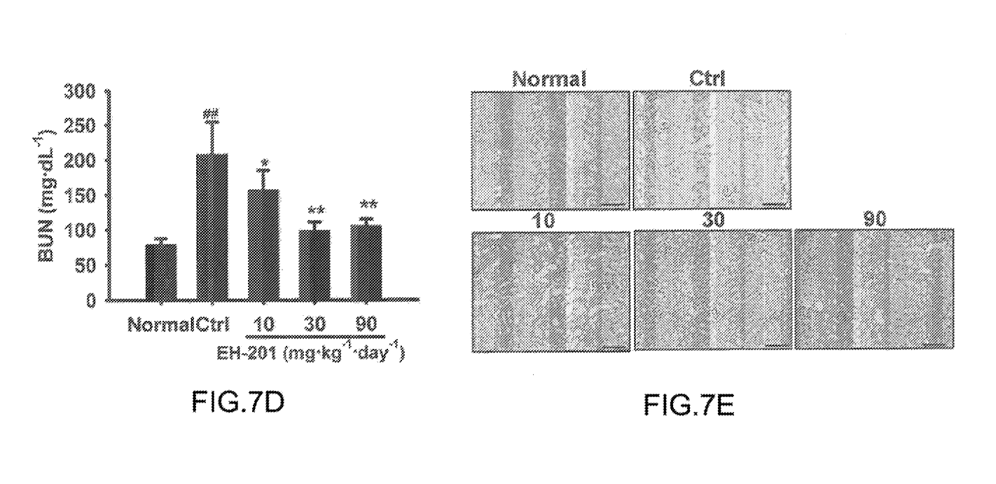

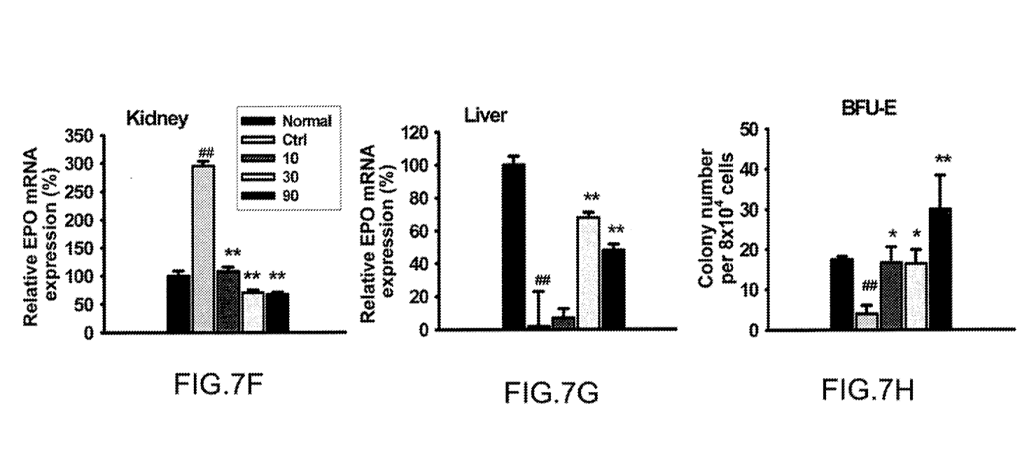

Since acute kidney injury may result from renal ischemia caused by the use of nephrotoxic agents, to examine the effect of EH-201-induced EPO production on the anaemia with renal insufficiency, an established cisplatin-induced nephropathy mouse model was adopted (FIG. 7A). Significant anaemia from day 10 and impaired renal function from day 13 after the first injection of cisplatin was observed (FIG. 7B and 7C). Notably, the administration of 30 and 90 mg/kg of EH-201 for 2 weeks (on day 28, FIG. 7B) led to an almost complete recovery of anaemia. Moreover, the BUN levels of the EH-201 30 and 90 mg/kg treatment groups were also significantly recovered (FIG. 7D). The histochemical examination revealed renal tubuloepithelial necrosis, vacuolation, and desquamation from day 13; however, treatment with EH-201 significantly attenuated this renal damage (FIG. 7E). In addition, a significant increase in EPO in the kidneys of the anaemic mice, and EH-201 treatment did not lead to any further increases were observed (FIG. 7F), a finding which may due to the compensatory effect of the remaining functional kidney cells and the recovered renal function generated by EH-201 relieving the hypoxic stress on the kidney. The EH-201 30 and 90 mg/kg treatments induced significant recovery of the hepatic EPO expression (FIG. 7G). Furthermore, EH-201 administration also activated the erythroid progenitor cells in the bone marrow (FIG. 7H). Collectively, these findings show that EH-201 improved the recovery from cisplatin-induced anaemia and renal dysfunction by inducing the production of EPO.

### (8) EH-201 Increases a Cellular EPO Expression Level in Mice RPE Cells

FIG. 18 shows EH-201 induction of cellular EPO expression level in mice RPE cells. C57mice RPE cells were incubated with 0.4, 2, 10 μg/ml EH-201 in DMEM supplemented with 10% FCS. The cultures were incubated at 37° C. for 24 hours. After incubation period, whole cell lysates were prepared with lysis buffer. Total cell lysates were prepared and subjected to western blot analysis to detect the level of endogenous EPO. GAPDH was used as a loading control. Bars represent mean±SD (n=3 different experiments; **p<0.01,***p<0.001).

### EXAMPLE 3

### Activating Mitochondrial Function and Haemoglobin Expression in Neuronal Cells by the Compound of the Present Invention

This example describes various assays that are useful in evaluating the activation of mitochondrial function and haemoglobin expression in neuronal cells by the compound of the present invention. The compound of the present invention is prepared according to the methods provided in Example 1. The potency of this compound is evaluated using a series of activity assays and these assays are further described in detail below.

### 1. Cell Culture

Astrocyte-enriched cultures were prepared from one-day-old C57BL/6J mice obtained from the Animal Center at the National Yang Ming University as described below. Briefly, cortical tissue was digested with trypsin, and the resultant dissociated cells were suspended in DMEM containing 10% FBS and incubated in 100-mm culture dishes. After 3 days in culture, the media was replaced with fresh 10% FBS/DMEM, and the cells were maintained at 37° C. for an additional 3 days. The cells were dissociated with trypsin, suspended in 10% FBS/DMEM and incubated in a 10-cm dish for 7-8 days prior to use. Cells prepared by this method consisted of approximately 90-95% astrocytes as determined by immunochemical staining with an antibody against glial fibrillary acidic protein (GFAP), a specific marker for astrocytes. Rat PC12 neuronal cells were maintained in RPMI 1640.

### 2. RNA Isolation and Real Time PCR

RNA was prepared using RNA-BeeTM RNA isolation reagent (Tel-test, Friendswood, Tex.). An aliquot of 5 jig total RNA was incubated with AMV-RT (Promega) to produce the cDNA for the RT-PCR analysis of the expression levels of β-actin, NGF and PGC-1α using the ABI Prism 7700 Sequence Detection System and the SYBR Green Master Mix kit (Applied Biosystems, Foster City, Calif.). The expression level of mouse β-actin was used as an internal reference. Relative gene expression levels were calculated with the 2-ΔΔCT method. Fragments (100-250 bp) were amplified using specific primers for each gene. The following primers were used: EPO (5′-AAT GGA GGT GGA AGA ACA GG-3′ and 5′-ACC CGA AGC AGT GAA GTG A-3′), Hb-β (5′-TGA TGC TGA GAA GGC TGC TGT CTC TG-3′) and (5′-GTG CCC TTG AGG CTG TCC AAG TGA-3′), PGC-1α (5′-AGC CGT GAC CAC TGA CAA CGA G-3′) and (5′-GCT GCA TGG TTC TGA GTG CTA AG-3′), HO-1(5′-CGC CTT CCT GCT CAA CAT T-3′) and (5′-TGT GTT CCT CTG TCA GCA TCA C-3′) and GAPDH (5′- TCT TCA CCA CCA TGG AGA AG-3′ and 5′-ACC AAA GTT GTC ATG GAT GAC-3′).

### 3. Western Blot

Cell and brain tissue lysates were prepared using a radioimmunoprecipitation assay lysis buffer. Approximately 20 μg of protein was loaded, and western blot analysis was performed using a monoclonal mouse antibody against EPO (1:500; sc-7956, Santa Cruz, California, USA), Hb-β (1:500; sc-31116, Santa Cruz, Calif., USA) and an anti-GAPDH antibody (1:10,000; ab9385, Abcam, Cambridge, UK) that was used as a loading control. A horseradish peroxidase-conjugated anti-IgG secondary antibody was used for enhanced chemiluminescence detection (Amersham, Buckinghamshire, UK).

### 4. Succinate Dehydrogenase Assay

Astrocytes or PC12 neuronal cells were plated at 10⁴ cells per well in 96-well plates. Twenty-four hours later, the cells were incubated with or without EPO or EH-201-containing media (100 μl per well) for 48 hours. Succinate dehydrogenase activity was determined by the MTT reduction assay. The activity was normalized to the cellular protein level (measured with a BioRad protein kit), and changes in absorbance were measured using a microplate reader (PerkinElmer Life Sciences Wallac Victor2). Activity was expressed relative to the control condition.

### 5. Intracellular Reactive Oxygen Species Generation

Astrocytes and PC12 neuronal cells were treated with EPO or EH-201 for 24 hours. The culture medium was replaced with 100 μM H₂O₂, and cells were incubated for 6 hours (astrocytes) or 30 minutes (PC12 cells). Reactive oxygen species (ROS) production in cells was then measured using 2′,7′-dichlorofluorescin diacetate (DCFH-DA; Molecular Probes, Eugene, Oreg., USA). DCFH-DA accumulates in cells and is hydrolyzed by cytoplasmic esterases to become 2′,7′-dichlorofluorescin. 2′,7′-Dichlorofluorescin is oxidized by H₂O₂ to give a fluorescent product, 2′,7′-dichlorofluorescein. Briefly, cultures in 96-well plates were washed with DMEM containing 1% FCS and loaded with 50 μM DCFH-DA for 30 minutes at 37° C. Wells were then washed twice with Kreb's buffer, and the cells were solubilized with 0.1 N NaOH in 50% methanol. The wells were vortexed for 10 minutes, and 2′-7′-dichlorofluorescein (DCF) fluorescence was either observed under fluorescence microscopy or measured in a microplate reader (PerkinElmer Life Sciences Wallac Victor2).

### 6. H₂O₂ Induced Cytotoxicity in Astrocytes and PC12 Neuronal Cells

Astrocytes and PC 12 neuronal cells were treated with EPO or EH-201 for 24 hours. Astrocyte culture medium was replaced with 500 μM H₂O₂, and the cells were incubated for 6 hours. PC12 cell culture medium was replaced with 250 μM H₂O₂, and the cells were incubated for 4 hours. Cell viability was determined by the exclusion of trypan blue as assessed by light microscopy.

### 7. Sleep Deprivation Procedure

Forty 12-week-old C57B1/6J adult male mice were obtained from the National Laboratory Animal Center (Taipei, Taiwan). Mice were housed at a constant temperature and supplied with laboratory chow (PMI, Brentwood, Mo., USA) and water ad libitum. The experimental procedure was approved by the Animal Research Committee of National Yang-Ming University. The animals were deprived of sleep (SD) or maintained in their home cages (control group) in the same room. Briefly, C57BL/6J male mice (7 weeks of age) were housed on a 12 hours/12 hours light/dark schedule with lights on at AM 6:00 and were handled for 7 days. The mice were sleep-deprived in their home cages for 5 hours by gentle handling beginning at AM 6:00 or left undisturbed (non-sleep-deprived mice). Mice were fed with normal diet or normal diet containing different concentrations of EH-201 (50, 100 or 200 mg/kg per day) for 3 days prior to sleep deprivation.

### 8. Passive Avoidance Task

Passive avoidance experiments were conduced as previously described with minor modifications. A two-way shuttle-box with a guillotine door placed between the modular testing chambers was employed. One chamber was illuminated with a 40 W bulb while the other remained in the dark. In the training session, the animals were individually placed in the illuminated chamber that faced away from the guillotine door. When the animal entered the darkened chamber, the door was silently lowered and a 0.5 mA foot shock was applied for 2 seconds through the grid floor. In the test sessions, the animals were again placed in the illuminated chamber, but no foot shock was applied. Latency to step through was recorded in each session.

### 9. Statistical Analysis

All results are expressed as the mean and standard deviation (SD). The significance of the differences of the means between more than two groups was determined using a one-way analysis of variance (ANOVA) followed by Tukey's post-hoc test. The Student's t-test was employed for the statistical comparison of paired samples. A P value of <0.05 was considered statistically significant.

### 10. Results

### (1) EH-201 Induced Neuronal EPO and Elevated Expression of EPO in Primary Astrocytes and PC12 Neuronal Cells

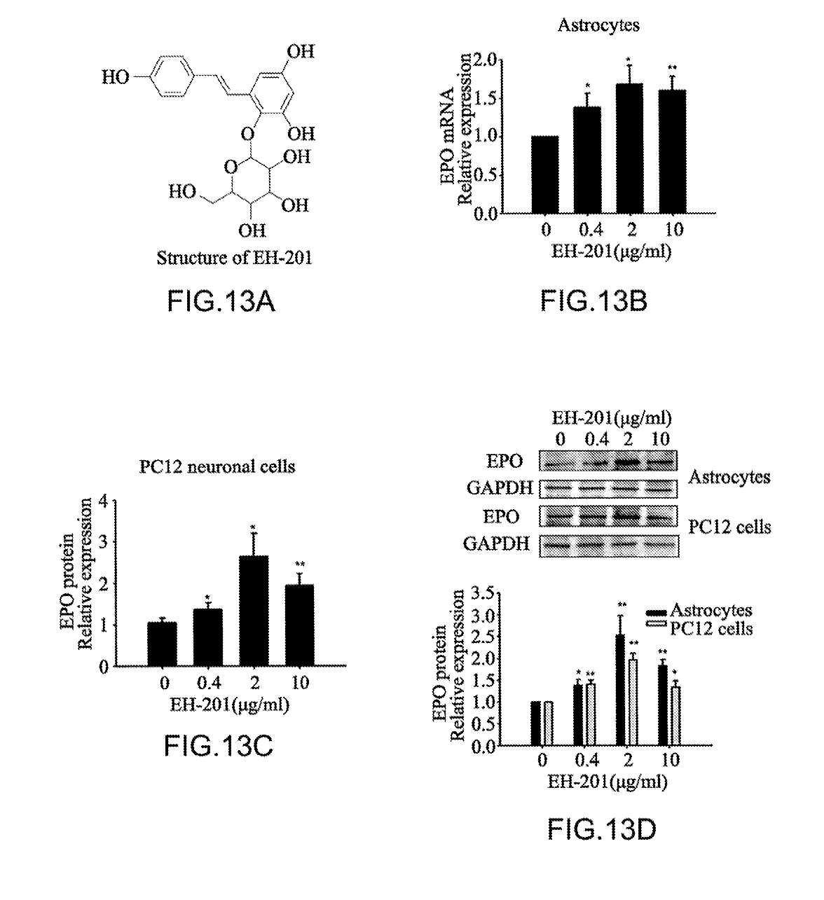

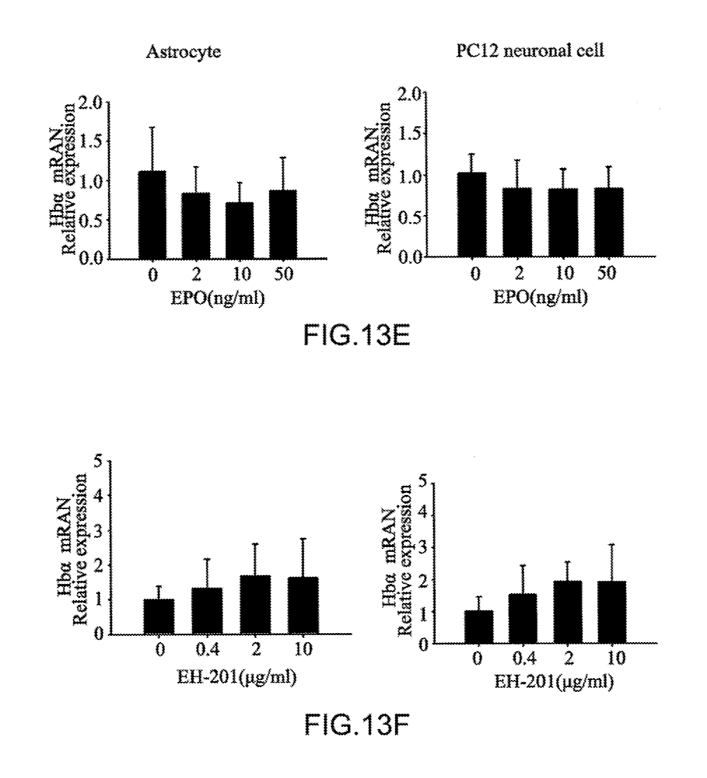

EH-201, a neuronal EPO inducer, elevated the expression of EPO in primary astrocytes and PC12 neuronal cells. Because exogenous EPO cannot cross the blood-brain barrier, its clinical use is limited. Thus, the effect of an endogenous neuronal EPO inducer, EH-201, was tested. The structure of EH-201 is shown in FIG. 13A. After EH-201 treatment, astrocytes demonstrated a dose-dependent increase in EPO mRNA expression, as measured by real time PCR analysis (FIG. 13B). EH-201 treatment also up-regulated EPO mRNA expression in PC12 neuronal cells (FIG. 13C). The intracellular EPO protein expression in astrocytes and PC12 cells was up-regulated during EH-201 treatment (FIG. 13D).

### (2) EH-201 Elevated the Expression of Mitochondrial Regulator PGC-1α and Hb in Primary Astrocytes and PC12 Neuronal Cells

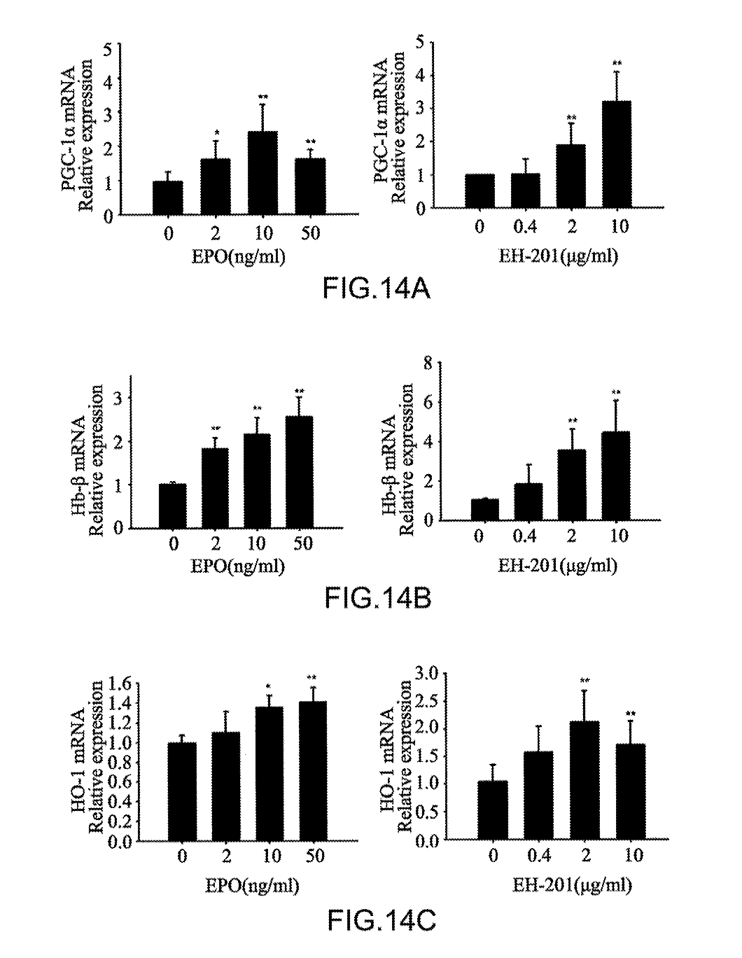

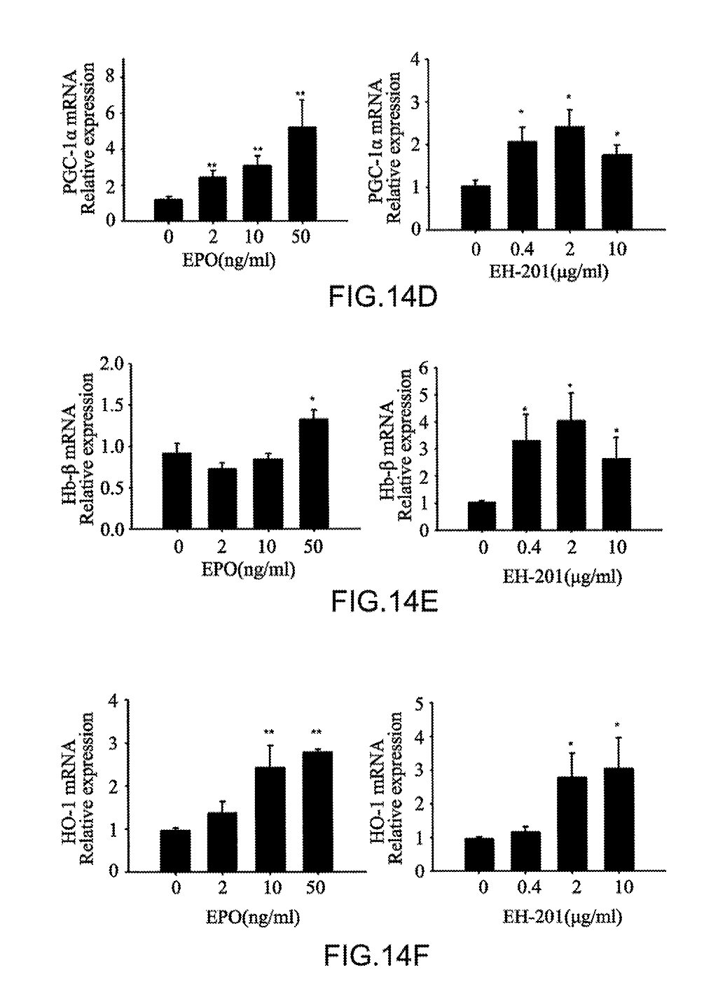

After EPO or EH-201 treatment for 24 hours, cellular mRNA was extracted to determine EPO-mediated gene expression. Real time PCR revealed elevated expression of PGC-1α and Hb-β mRNA expression; HO-1, a known antioxidant gene up-regulated by EPO, was also induced during EH-201 treatment, both in astrocytes (FIG. 14A to FIG. 14C) and in PC12 neuronal cells (FIG. 14D to FIG. 14F); Hb-α expression, however, was not significantly changed (FIG. 13E to FIG. 13F).

### (3) EH-201 Increased Mitochondrial Activity and Attenuated Oxidative Stress in Primary Astrocytes and PC12 Neuronal Cells

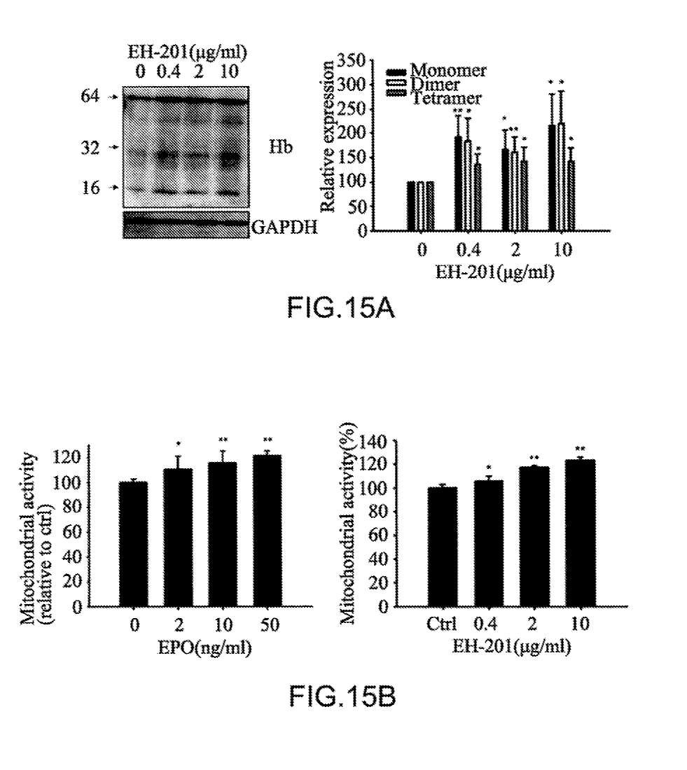

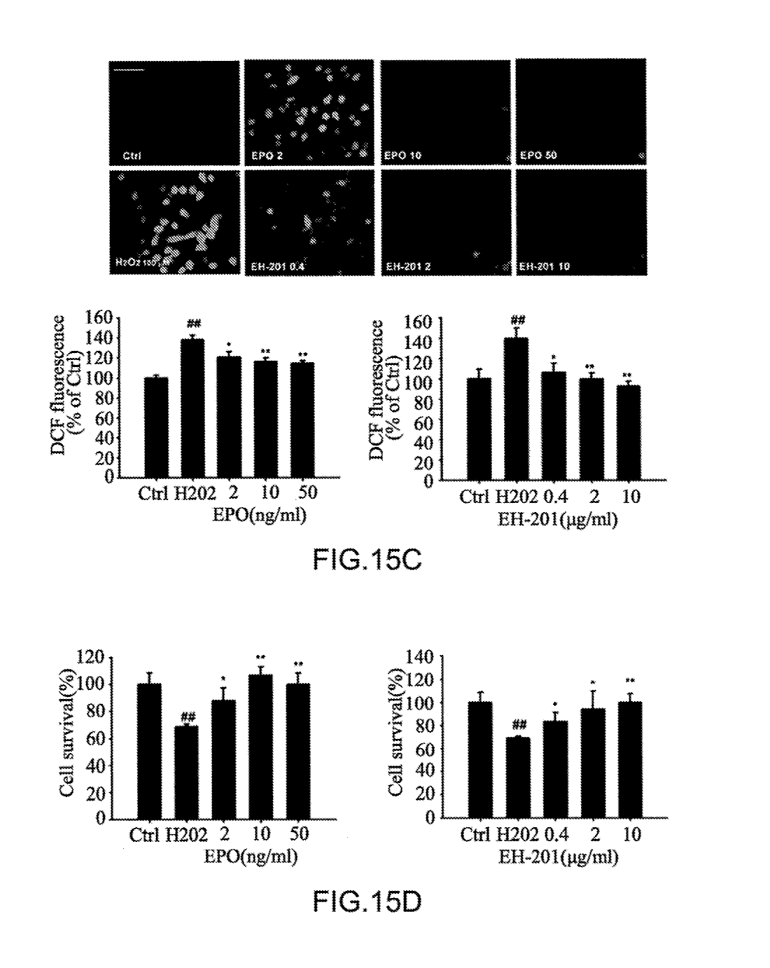

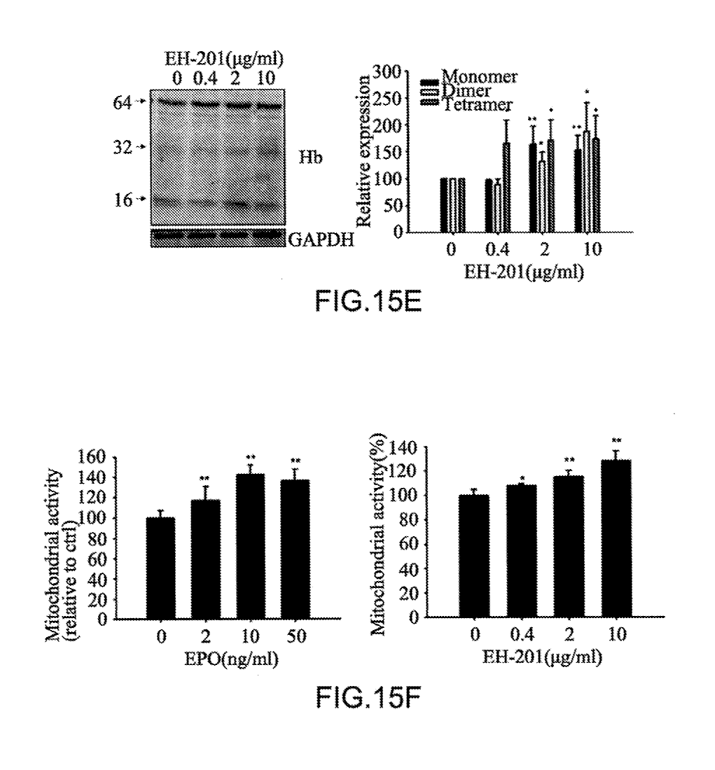

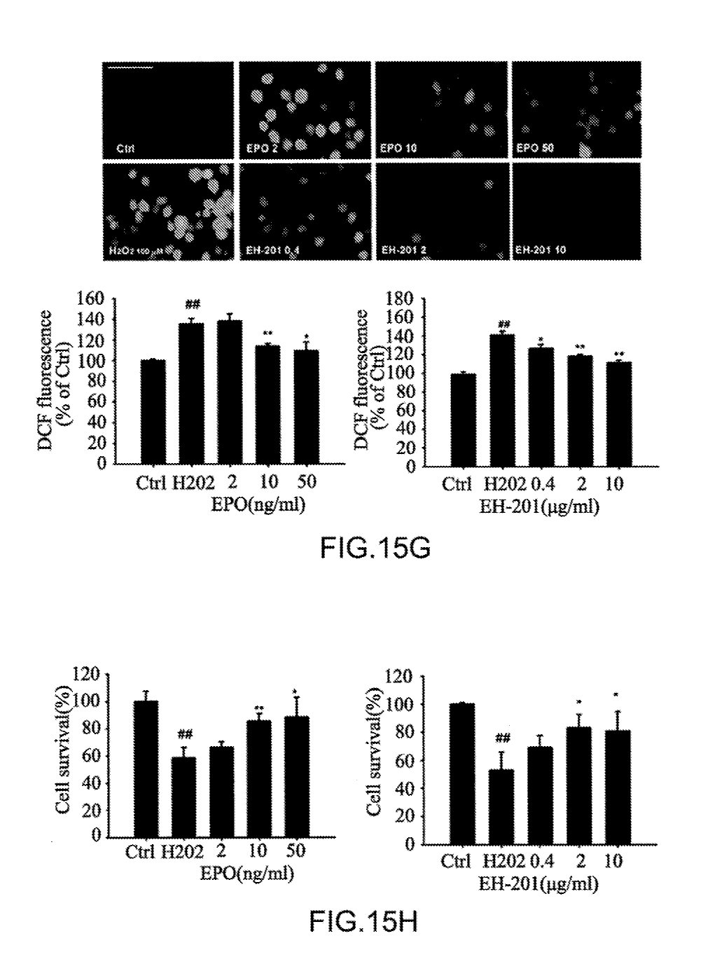

Because PGC-1α and Hb are known as mitochondrial regulators, it was analyzed which form of Hb was regulated by EH-201. FIG. 15A and E showed that EH-201 increased all three forms of Hb expression in astrocytes and PC12 cells. Next, mitochondrial activity in cells treated with or without EPO or EH-201 was measured by the MTT assay. FIG. 15B and F showed that EPO or EH-201 induced mitochondrial activity in both astrocytes and PC12 cells. It was examined whether EPO or EH-201-mediated up-regulation of these genes attenuates oxidative stress induced by H₂O₂ in astrocytes and PC12 cells. It was estimated ROS generation in the cultured cells after exposure to H₂O₂ using an oxidative probe, CM-H2DCFDA. EPO and EH-201 treatment decreased intracellular ROS in astrocytes (FIG. 15C) and PC12 cells (FIG. 15G). EPO and EH-201 also decreased cell toxicity in cells exposed to H₂O₂, indicating that the mitochondrial regulation and ROS homeostasis effect of EPO is biologically important (FIGS. 15D and 15H).

### (4) EPO is Required for EH-201-Mediated Increased Mitochondrial Activity and Attenuation of Oxidative Stress in Primary Astrocytes and PC12 Neuronal Cells

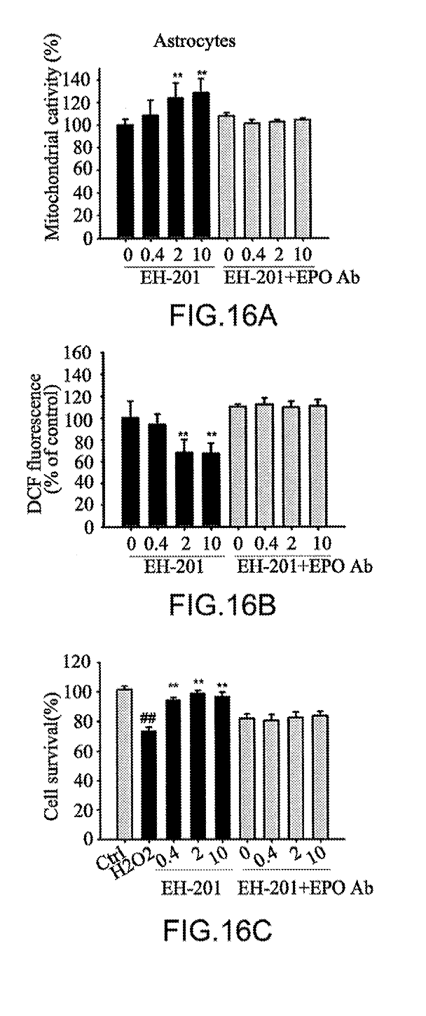

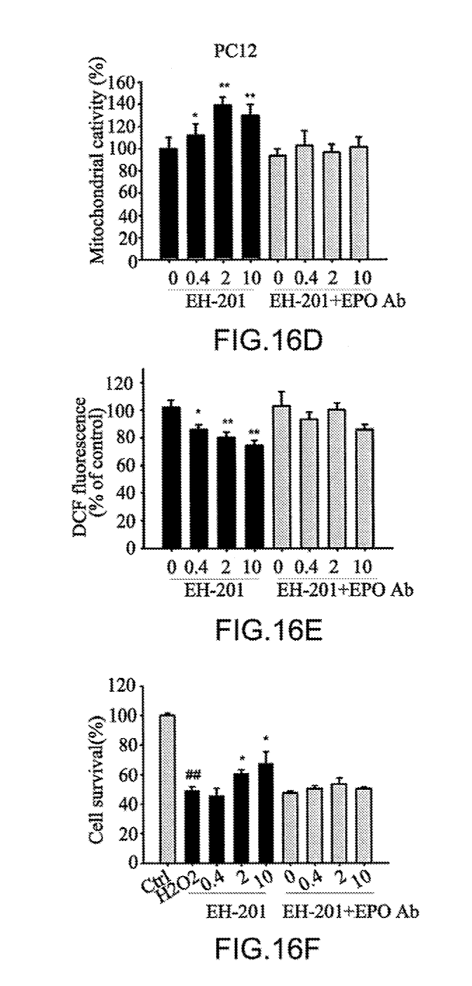

It was evaluated whether the increased mitochondrial activity and the reduction in H₂O₂-induced ROS generation and cytotoxicity following treatment with EH-201 in astrocytes and PC12 cells were dependent on EPO. The increased mitochondrial activity observed with EH-201 treatment was blocked in the presence of an anti-EPO antibody as measured by the MTT assay (FIG. 16A) compared to cells treated with EH-201 alone. The reduction of ROS generation induced by H₂O₂ in astrocytes and PC12 cells treated with EH-201 was abolished when cells were co-incubated with an anti-EPO antibody (FIG. 16B). The anti-EPO antibody also inhibited the EH-201-mediated reduction in H₂O₂-inducted cytotoxicity (FIG. 16C).

### (5) Effects of EH-201 in a Mouse Model of Sleep Deprivation-Induced Memory Loss

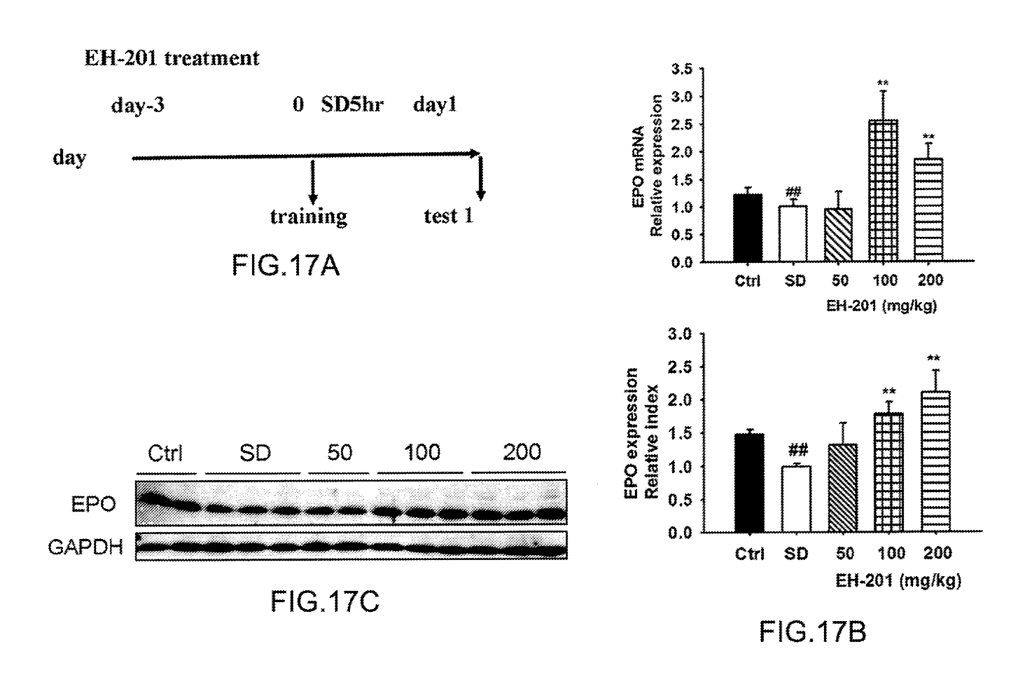

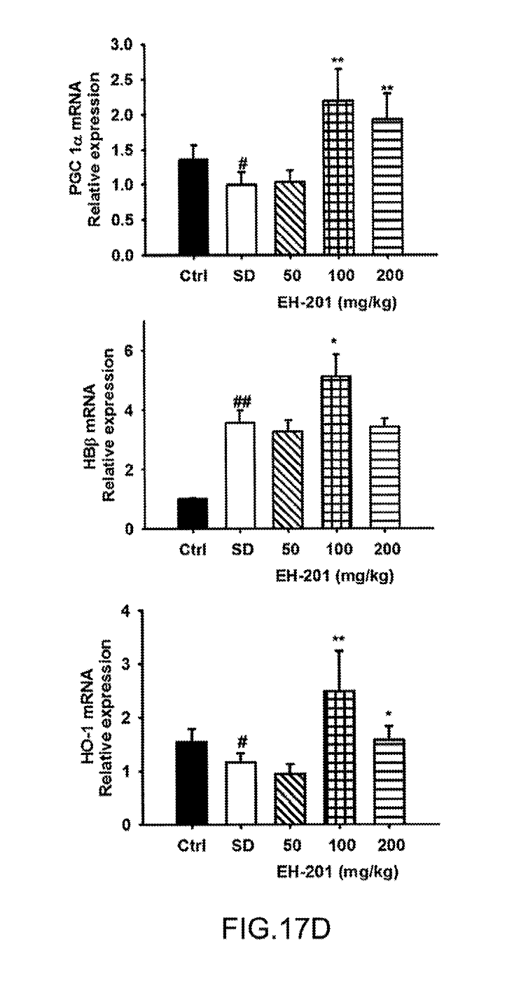

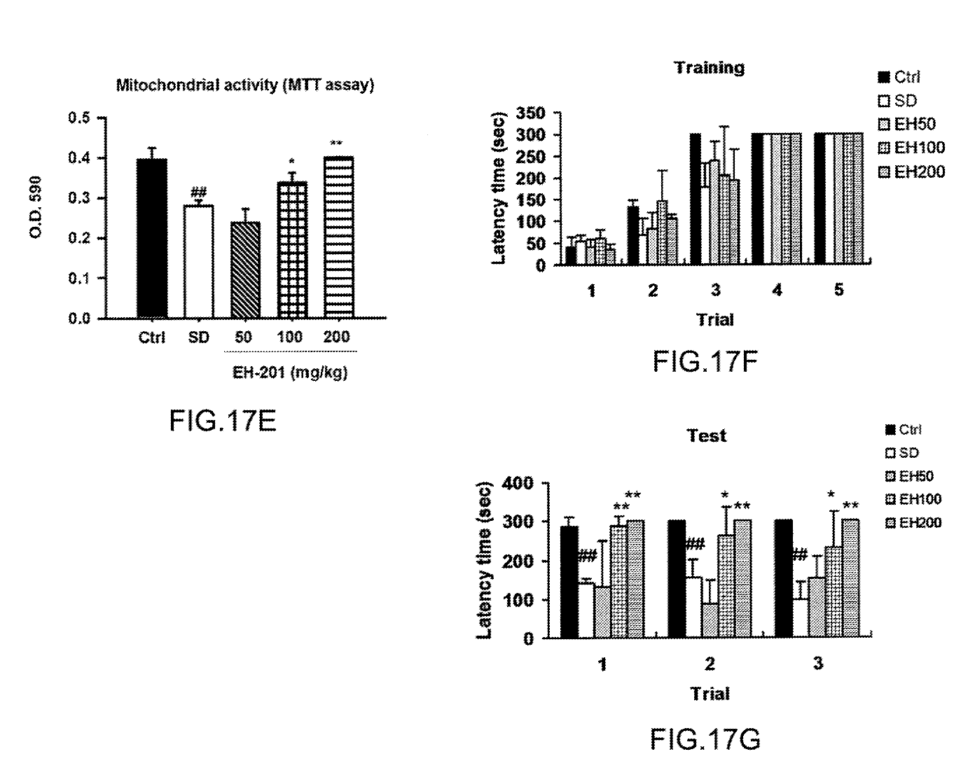

It was evaluated the neuroprotective effect of EH-201 on memory by using a SD model. The experimental procedure is outlined in FIG. 17A. It was analyzed the EPO expression in the hippocampus from each treated animal. Real time PCR and western blotting showed an increase in EPO expression in animals fed with EH-201 (FIG. 17B and 17C). Hbβ, HO-1 and PGC-1α mRNA expression in the hippocampus was analyzed by real time PCR (FIG. 17D). It was further evaluated the mitochondrial succinate dehydrogenase activity using the MTT assay (FIG. 17E). In the passive avoidance test, animals fed EH-201 for three days did not exhibit a difference in the ability to learn (FIG. 17F). However, there was a significant improvement in memory performance in EH-201-fed mice after SD in the passive avoidance test (FIG. 17G). The gene expression changes and the increased mitochondrial activity in hippocampus from animals fed with EH-201, especially at doses of 100 and 200 mg/kg, correlated with the passive avoidance test.

### EXAMPLE 4

### Inducing Autophagy by the Compound of the Present Invention

This example describes various assays that are useful in evaluating the inducing autophagy by the compound of the present invention. The compound of the present invention is prepared according to the methods provided in Example 1. The potency of this compound is evaluated using a series of activity assays and these assays are further described in detail below.

### 1. POS Phagocytosis Assays

After the treatments indicated above, cells were treated with FITC-OS (1×10⁷ OS/well) and incubated at 37° C. for 4 hours. Untreated cells were used to obtain baseline fluorescence. The cells were washed four times with (EBSS) to remove excess POS. Finally, EBSS was added to each well, at 100 μl/well, and the analysis of mean FITC-OS fluorescence was achieved by a fluorometer, which quantified the FITC-OS fluorescence at excitation 485 nm and emission 535 nm. Thereafter, fluoro-quenching dye was added per well, at 25 μl/well, and the dye was incubated at 37° C. for 30 minutes; the dye was quantified by fluorometer analysis of fluorescence (excitation, 485 nm: emission, 535 nm).

### 2. Western Blot Analysis

After the indicated treatments, cells were washed twice with ice-cold PBS, and were lyzed in extraction buffer (1M Tris, pH 6.8, 10% SDS, 1M DTT). 10-15μg of total protein was separated by SDS-PAGE, and analyzed by immunoblotting using chemiluminescence. The primary antibodies used were LC3B antibody (Gene Tex, USA, 1:1000), EPO (GAPDH (Santa Cruz, Calif., USA,1:1000) or GAPDH (Santa Cruz, Calif., USA,1:1000), peroxidase-conjugated anti-mouse IgG or peroxidase-conjugated anti-rabbit IgG. (Santa Cruz, Calif., USA, 1:1000). The intensity of protein bands was quantified using image j software and the ratio of specific band to control was analyzed.

3. Labeling of Autophagic Vacuoles with Monodansylcadaverine

Monodansylcadaverine (MDC) is a spontaneously fluorescent dye that can be incorporated selectively into autophagosomes and autolysosomes. Cells were incubated with 0.05 mM MDC in PBS at 37° C. for 1 hour. After incubation, cells were washed two times with PBS and immediately analyzed by fluorescence microscopy (excitation: 380-420 nm, barrier filter 450 nm).

### 4. Cell Culture and Treatment

The culture of murine kidney slices and primary mice hepatocytes have described previously. These cultures were treated with EH-201 at different doses (0.6, 2.5, 10 and 40 mg/ml), autophagy activator rapamycin (Rm, 50 nM) or autophagy inhibitor 3-methyladenine (3MA, 10 mM) for 24 hours. The hepatocyte culture under starvation (sty) was autophagy activation control.

### 5. Results

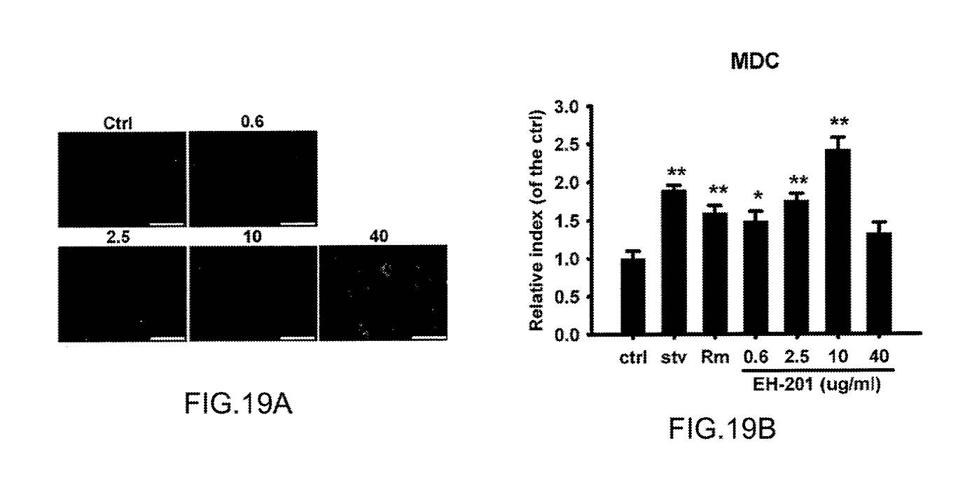

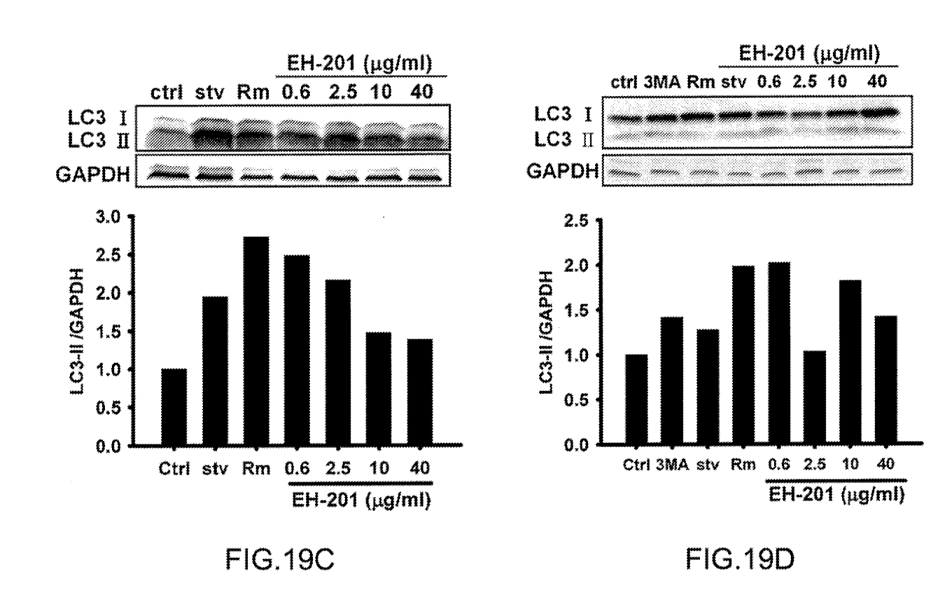

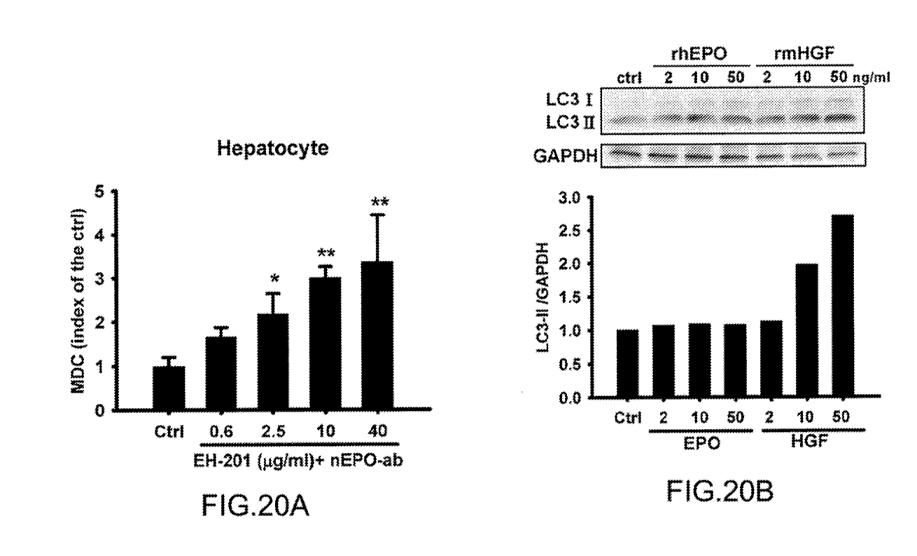

(1) FIG. 19A to FIG. 19D show induction of autophagy by EH-201.
(2) As shown in FIG. 20A and FIG. 20B, EH-201 induced autophagic activation is through hepatocyte growth factor (HGF) induction.

The foregoing descriptions are only illustrative of the features and functions of the present invention but are not intended to restrict the scope of the present invention. It is apparent to those skilled in the art that all equivalent modifications and variations made in the foregoing descriptions according to the spirit and principle in the disclosure of the present invention should fall within the scope of the appended claims.

1. A method for treating insomnia through inducing autophagy, the method comprising administering to the subject a therapeutically effective amount of a compound of formula (I): 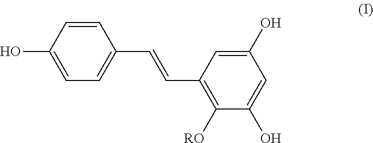
wherein R is a glycosyl group, and the therapeutically effective amount is an amount effective in inducing erythropoietin (EPO)-mediated haemoglobin (Hb) expression in a nonhaematopoietic cell of the subject.

2. The method of claim 1, wherein the glycosyl group is one selected from the group consisting of dihydroxyacetone, glucose, galactose, glyceraldehyde, threose, xylose, mannose, ribose, ribulose, tagatose, psicose, fructose, sorbose, rhamnose, erythrose, erthrulose, arabinose, lyxose, allose, altrose, gulose, idose, talose, sucrose, lactose, maltose, lactulose, trehalose, cellobose, isomaltotriose, nigerotriose, maltotriose, melezitose, maltotriulose, raffinose, kestose, and a combination thereof.

3. The method of claim 1, wherein the compound induces Hb-α, Hb-β, or dimeric Hb expression in the nonhaematopoietic cell of the subject.

4. The method of claim 1, wherein the compound enhances endogenous EPO expression and stimulates Hb expression in the nonhaematopoietic cell of the subject.

5. A method for treating insomnia through inducing autophagy, the method comprising administering to the subject a therapeutically effective amount of a compound of formula (I): 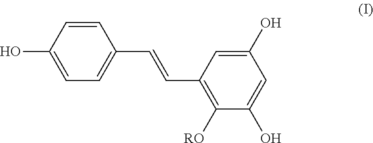
wherein R is a glycosyl group, and the therapeutically effective amount is an amount effective in inducing erythropoietin (EPO)-mediated mitochondrial biogenesis in a nonhaematopoietic cell of the subject.

6. The method of claim 5, wherein the glycosyl group is one selected from the group consisting of dihydroxyacetone, glucose, galactose, glyceraldehyde, threose, xylose, mannose, ribose, ribulose, tagatose, psicose, fructose, sorbose, rhamnose, erythrose, erthrulose, arabinose, lyxose, allose, altrose, gulose, idose, talose, sucrose, lactose, maltose, lactulose, trehalose, cellobose, isomaltotriose, nigerotriose, maltotriose, melezitose, maltotriulose, raffinose, kestose, and a combination thereof.
